(19) United States
(12) Patent Application Publication
(10) Pub. No.: US 2026/0301958 A1
(43) Pub. Date: Oct. 1, 2026

(54) AI-DRIVEN DIAGNOSIS AND TREATMENT OF PERIPHERAL ARTERY DISEASE

(71) Applicant: GuideAl Health, Inc., Boston, MA (US)

(72) Inventors: Raj Pulin Shah, Tavares, FL (US); Luke James Higgins, Orleans, MA (US); Kourosh Jafari-Khouzani, Orange, CA (US); Amin Katouzian, Lexington, MA (US)

(21) Appl. No.: 19/631,887

(22) Filed: Mar. 27, 2026

Related U.S. Application Data
(60) Provisional application No. 63/779,793, filed on Mar. 28, 2025.

(51) Int. Cl. G16H 50/20; G16H 30/20

(52) U.S. Cl. CPC G16H 50/20; G16H 30/20

(57) ABSTRACT

Provided herein are methods and devices for diagnosing peripheral artery disease (PAD) using computed tomography angiography (CTA) images. The method involves obtaining at least one CTA image of the patient's vascular system and inputting this image into a trained machine learning model. The model analyzes the image to generate groups of images that highlight vascular abnormalities, including segmented and annotated images. These generated images are then matched to reference images with known characteristics associated with PAD. Based on this analysis, a diagnosis is confirmed, indicating the presence and severity of PAD. Additionally, a comprehensive report is generated, detailing the diagnosis, severity of the condition, and recommendations for treatment options. This approach enhances diagnostic accuracy and efficiency, facilitating timely clinical decision-making for patients with PAD.

### CROSS REFERENCE TO RELATED APPLICATION

This application claims priority to U.S. Provisional Patent Application No. 63/779,793, filed on Mar. 28, 2025, entitled “AI-Driven Diagnosis of Peripheral Artery Disease,” the entire contents of which are incorporated by reference herein.

### TECHNICAL FIELD

This application relates generally to computing systems, and in particular to computing systems for artificial intelligence (AI)-driven analysis of image and other data related to diseases.

### BACKGROUND

Medical imaging for vascular analysis can utilize computer generated images to visualize the vascular system. In some instances, these images are reviewed and analyzed by radiologists.

### SUMMARY

The present disclosure provides methods and systems for analyzing medical imaging data using machine learning techniques to enhance the diagnosis and treatment planning of vascular conditions. Methods and systems of the present disclosure may be used for the diagnosis of peripheral artery disease (PAD), quantification of vascular conditions, visual representation of imaging data, comprehensive report generation, and assisting in treatment planning.

In one aspect, the present disclosure provides a method for diagnosing a patient having peripheral artery disease (PAD). In some embodiments, the method includes using at least one computer processor to perform obtaining at least one computed tomography angiography (CTA) image of at least a portion of the patient, where the at least one CTA image of the at least a portion of the patient include at least a portion of a vascular system; providing the at least one CTA image of the at least a portion of the patient as input to a machine learning model, where the machine learning model is a trained machine learning model trained by a database of at least one reference image with known characteristics associated with PAD; classifying characteristics of the at least a portion of the vascular system based on the at least one CTA image of the at least a portion of the patient using the trained machine learning model, where the trained machine learning model is configured to characterize the at least a portion of the vascular system; determining a diagnosis of the patient based on the classified characteristics of the at least a portion of the vascular system from the at least one CTA image of the at least a portion of the patient, where the diagnosis includes at least one condition of the patient correlated to at least one indicator of PAD; and generating a report based on the diagnosis, where the report includes a diagnosis of PAD, a severity of PAD, and/or recommendations for treatment options.

In some embodiments, a classification of the characteristics includes generating one or more groups of images from the at least one CTA image based on characteristics associated with PAD. In some embodiments, the generating one or more groups of images includes segmented images highlighting vascular abnormalities, annotated images, and/or comparative images. In some embodiments, a classification of the characteristics includes matching images from the one or more groups of images to the at least one reference image with known characteristics associated with PAD, where a process of matching images is conducted by the trained machine learning model. In some embodiments, the classified characteristics of the at least a portion of the vascular system are obtained from one or more generated groups of images. In some embodiments, the characteristics associated with PAD are associated with a blockage in a vessel, where a cross sectional area of the blockage area is at least about 50% of a cross sectional area of the vessel. In some embodiments, a loss function is employed to train the machine learning model to minimize an error in classification of characteristics associated with PAD. In some embodiments, the loss function uses binary cross-entropy loss, where the binary cross-entropy loss is below 0.1.

Reducing errors through high model accuracy serves as an optimization filter that enhances the efficiency of the entire computational pipeline. By minimizing false positives and inaccuracies, the system can bypass redundant operations, starting with early termination at specific quality control checkpoints. If the selection or quality criteria are not met, the study is immediately rejected, which prevents the computer processor—such as a GPU, CPU, or TPU—from wasting processing cycles and power on unviable data. Furthermore, accurate landmark identification enables the system to establish a processing range with better accuracy. This ensures that intensive deep-learning computations, such as those performed by the Multi-head U-Net (MHU), are focused strictly on relevant anatomical segments rather than the entire 3D volume, thereby significantly lowering the total computational burden.

Memory requirements are similarly reduced through the precise pruning of false positive regions and the elimination of orphaned nodes. The system can employ graph-based segmentation pruning to identify the most probable vessel path, and by removing nodes that are not connected to the primary vascular structure, it minimizes the size of the 3D graph representation held in the memory unit. Additionally, accurate axial series identification and quality assessment ensure that only the best series is loaded into active memory, preventing the storage of redundant or low-quality DICOM image stacks. This high degree of accuracy also improves latency by reducing the reliance on intensive post-processing steps, such as complex noise reduction or manual expert corrections. Because the model's classifications are closely aligned with true target values through the use of an optimized loss function, the diagnostic results move more rapidly from organization to final report creation.

Finally, reducing errors directly optimizes communication bandwidth by enabling the use of compact numerical representations instead of raw data. Because the Vision Transformer (ViT) and trained models are highly accurate, they can effectively transform bulky 3D CTA volumes into streamlined embeddings. These transformed representations capture desired features while requiring significantly fewer bits for transmission via the communication interface. Bandwidth efficiency is further protected by the early and accurate detection of imaging artifacts, such as motion or beam hardening, which prevents the transmission of corrupted or useless data packets to remote workstations or cloud-based storage.

In some embodiments, obtaining the at least one CTA image includes receiving at least one Digital Imaging and Communications in Medicine (DICOM) image, where the DICOM image are associated with the patient having PAD. In some embodiments, the machine learning model includes a Vision Transformer (ViT) model. In some embodiments, the ViT model is configured to process maximum intensity projections (MIP) segments of 3D CTA volumes, where the MIP segments include numerically transformed representations of local and/or global features derived from the 3D CTA volumes. In some embodiments, the ViT model is configured to analyze the 3D CTA volumes and generate regional heatmaps, where the regional heatmaps are attributed to vascular abnormalities. In some embodiments, the regional heatmaps are superimposed on the 3D CTA volumes. In some embodiments, the at least one DICOM image is analyzed for at least one associated tag. In some embodiments, the at least one associated tag is coupled with at least one image among the at least one DICOM image generating at least one tagged DICOM image. In some embodiments, the at least one DICOM image is organized into coherent series by the machine learning model. In some embodiments, the machine learning model is configured to create a structured database of the at least one tagged DICOM image.

In some embodiments, the machine learning model is configured to create a primary classification of the at least one tagged DICOM image. In some embodiments, the primary classification of the at least one tagged DICOM image include an identification of axial series. In some embodiments, the identification of axial series includes evaluating a thickness of a slice of the at least one image among the at least one DICOM image. In some embodiments, the identification of axial series includes assessing an anatomical coverage. In some embodiments, the identification of axial series includes evaluating quality metrics of the at least one tagged DICOM image. In some embodiments, the trained machine learning model is configured to automatically identify and label anatomical structures, where the anatomical structures include aorta, common iliac arteries, external right and left iliac arteries, left common femoral arteries, right common femoral arteries, left superficial femoral arteries, right superficial femoral arteries, left popliteal arteries, right popliteal arteries, left anterior tibial artery (ATA) bifurcation, or right ATA bifurcation. In some embodiments, the trained machine learning model is configured to optimize a processing range of the automatically identification and labeling of the anatomical structures. In some embodiments, the trained machine learning model is configured to identify medical devices within the at least a portion of the vascular system associated with the at least one CTA image of the at least a portion of the patient, where the medical devices include stents or bypass grafts. In some embodiments, the trained machine learning model is configured to identify imaging artifacts within the at least a portion of the vascular system associated with the at least one CTA image of the at least a portion of the patient, where the imaging artifacts include motion artifacts, streak artifacts, or beam hardening artifacts.

In some embodiments, the trained machine learning model is configured to digitally segment regions within at least one image of the at least a portion of the vascular system associated with the at least one CTA image of the at least a portion of the patient, where segmented image include arterial walls, lesions, or thrombosis. In some embodiments, the trained machine learning model is configured to calculate an area ratio and/or percentage associated with stenosis. In some embodiments, the trained machine learning model is configured to classify a lesion's morphology, where the lesion is identified within the at least a portion of the vascular system associated with the at least one CTA image of the at least a portion of the patient. In some embodiments, the trained machine learning model is configured to provide a treatment guidance based on the classification of the lesion's morphology, where the treatment guidance is selected from a set of recorded treatments according to the classification of the lesion's morphology, where the recorded treatments are approved by certified clinicians.

In some embodiments, the trained machine learning model is configured to provide a guidance on selecting an intervention device based on the classification of the lesion's morphology and/or lesion's characteristics, where the guidance on selecting the intervention device is selected from a set of recorded guidance according to the classification of the lesion's morphology and/or the lesion's characteristics and where the recorded guidance is approved by certified clinicians. In some embodiments, the trained machine learning model is configured to generate a structured radiological report comprising at least one condition of the patient correlated to at least one indicator of PAD. In some embodiments, the trained machine learning model is configured to generate a structured radiological report comprising at least one lesion's morphology, anatomical landmarks, and the spatial distribution of the at least one lesion within the vascular system.

In some embodiments, the structured radiological report includes a 3D vascular mapping, detailed lesion characterization, quantitative measurements and/or treatment recommendations. In some embodiments, the trained machine learning model includes a neural network model. In some embodiments, the neural network model includes one or more single-head U-Net (SHU) and/or multi-head U-Net (MHU) convolutional layers. In some embodiments, the characteristics of lesions include hard lesions, soft tissue lesions or mixed lesions. In some embodiments, the segmented images include a segment correlated with a blockage in a vessel within the at least a portion of the vascular system. In some embodiments, the blockage in the vessel includes hard lesions, soft tissue lesions or mixed lesions. In some embodiments, the trained machine learning model is configured to identify arteries within the vascular system based on an analysis of at least one CTA image.

In some embodiments, the trained machine learning model is configured to distinguish between arteries and veins within the vascular system based on an analysis of at least one CTA image. In some embodiments, the trained machine learning model is configured to isolate at least one artery from a plurality of arteries identified within the vascular system based on an analysis of at least one CTA image. In some embodiments, the trained machine learning model is configured to generate a histogram representing the characteristics of lesions. In some embodiments, the trained machine learning model is configured to generate a 3D vascular map based on at least one CTA image. In some embodiments, the trained machine learning model is configured to recommend a treatment plan, where the treatment plan includes a recommended use of an interventional tool based on a 3D construction of lesions identified within the vascular system. In some embodiments, where the report generated by the method includes anatomical locations and/or dimensions of lesions.

In one aspect, the present disclosure provides a computing system comprising at least one processor; and at least one memory device including instructions embodied thereon, where the instructions, which when executed by the at least one processor, cause the processor to perform operations for training a machine learning model for analyzing medical conditions, the operations comprising obtaining a dataset comprising a plurality of computed tomography angiography (CTA) images, where the dataset includes images of a vascular system with known characteristics associated with at least one medical condition; preprocessing the obtained CTA images to standardize formats for analysis; segmenting anatomical structures within the CTA images using the machine learning model; extracting features from the segmented anatomical structures, where the features include characteristics associated with the at least one medical condition; training the machine learning model using extracted features enabling the model to recognize the at least one medical condition; validating the trained machine learning model against a separate validation dataset to assess its performance and accuracy; and iteratively refining the trained machine learning model based on validation results.

In one aspect, the present disclosure provides a computing system for diagnosing a patient having peripheral artery disease (PAD) comprising at least one processor; and at least one memory device comprising instructions, where the instructions, upon execution, cause the processor to perform operations for PAD diagnosis, the operations comprising obtaining at least one computed tomography angiography (CTA) image of at least a portion of the patient, where the at least one CTA image includes at least a portion of the vascular system of the patient; providing the at least one CTA image as input to a machine learning model, where the machine learning model is trained on a database of reference images with known characteristics associated with PAD; generating one or more groups of images from the at least one CTA image based on characteristics associated with PAD, where generated one or more groups of images include segmented images highlighting vascular abnormalities, annotated images, and/or comparative images; matching images from the one or more groups of images to the at least one reference image with known characteristics associated with PAD, where the matching process is conducted by the trained machine learning model; determining a state of the potential PAD condition from the at least one CTA image using the machine learning model for automated image analysis; determining a diagnosis of the patient based on the generated one or more groups of images, where the diagnosis includes at least one condition of the patient correlated to at least one indicator of PAD; and generating a report based on the diagnosis, where the report includes a diagnosis of PAD, an assessment of its severity, and/or recommendations for treatment options.

In one aspect, the present disclosure provides a method for diagnosing a patient having peripheral artery disease (PAD), the method comprising using at least one computer processor to perform obtaining at least one computed tomography angiography (CTA) image of at least a portion of the patient, where the at least one CTA image of the at least a portion of the patient include at least a portion of a vascular system; providing the at least one CTA image of the at least a portion of the patient as input to a machine learning model, where the machine learning model is a trained machine learning model trained by a database of at least one reference image with known characteristics associated with PAD; classifying characteristics of the at least a portion of the vascular system based on the at least one CTA image of the at least a portion of the patient using the trained machine learning model, where the trained machine learning model is configured to characterize the at least a portion of the vascular system with an accuracy of about 80%; determining a diagnosis of the patient based on the classified characteristics of the at least a portion of the vascular system from the at least one CTA image of the at least a portion of the patient, where the diagnosis includes at least one condition of the patient correlated to at least one indicator of PAD; and generating a report based on the diagnosis, where the report includes a diagnosis of PAD, a severity of PAD, and/or recommendations for treatment options.

Another aspect of the present disclosure provides a non-transitory computer readable medium comprising machine executable code that, upon execution by one or more computer processors, implements any of the methods above or elsewhere herein.

Another aspect of the present disclosure provides a system comprising one or more computer processors and computer memory coupled thereto. The computer memory includes machine executable code that, upon execution by the one or more computer processors, implements any of the methods above or elsewhere herein.

Additional aspects and advantages of the present disclosure will become readily apparent to those skilled in this art from the following detailed description, where only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference. To the extent publications and patents or patent applications incorporated by reference contradict the disclosure contained in the specification, the specification is intended to supersede and/or take precedence over any such contradictory material.

### BRIEF DESCRIPTION OF THE DRAWINGS

The features of the disclosure are set forth with particularity in the appended claims. A better understanding of the features and advantages of the present disclosure will be obtained by reference to the following detailed description that sets forth illustrative embodiments, in which the principles of the disclosure are utilized, and the accompanying drawings (also “Figure” and “FIG.” herein), of which:

FIG. 1 illustrates the workflow for processing medical imaging data to classify peripheral artery disease (PAD).

FIG. 2 illustrates that the Single-head U-Net (SHU) model was utilized for vessel wall segmentation in two-dimensional (2D) imaging data.

FIG. 3 illustrates that the Multi-head U-Net (MHU) model was employed for vessel wall and lesion segmentation in two-dimensional (2D) imaging data.

FIG. 4A and FIG. 4B illustrate tables presenting the performance of different models in terms of vessel wall (VW) segmentation and lesion segmentation.

FIGS. 5, 6, 7, 8, and 9 illustrate exemplary CTA images that represent the AI-generated vessel wall (VW) and lesion segmentation results compared to manually drawn borders by an expert.

FIG. 10A illustrates an exemplary overview of an exemplary system for classifying maximum intensity projections (MIPs) and 3D computed tomography angiography (CTA) volumes to identify the presence of peripheral artery disease. FIGS. 10B, 10C, and 10D illustrate exemplary modules for the systems presented in FIG. 10A

FIG. 11A illustrates an exemplary series of modules involved in the processing of medical imaging data for the classification and identification of peripheral artery disease. FIGS. 11B, 11C, 11D, 11E, 11F, 11G, and 11H illustrate exemplary sequential steps involved in the processing of the medical imaging data for the modules presented in FIG. 11A.

FIG. 12 shows a computer system that is programmed or otherwise configured to implement methods provided herein.

### DETAILED DESCRIPTION

While various embodiments of the disclosure have been shown and described herein, it will be obvious to those skilled in the art that such embodiments are provided by way of example only. Numerous variations, changes, and substitutions may occur to those skilled in the art without departing from the disclosure. It should be understood that various alternatives to the embodiments of the disclosure described herein may be employed.

Whenever the term “at least,” “greater than,” or “greater than or equal to” precedes the first numerical value in a series of two or more numerical values, the term “at least,” “greater than” or “greater than or equal to” applies to each of the numerical values in that series of numerical values. For example, greater than or equal to 1, 2, or 3 is equivalent to greater than or equal to 1, greater than or equal to 2, or greater than or equal to 3.

Whenever the term “no more than,” “less than,” or “less than or equal to” precedes the first numerical value in a series of two or more numerical values, the term “no more than,” “less than,” or “less than or equal to” applies to each of the numerical values in that series of numerical values. For example, less than or equal to 3, 2, or 1 is equivalent to less than or equal to 3, less than or equal to 2, or less than or equal to 1.

The terms “image,” “slice,” “radiographic image,” “tomogram,” or “cross-sectional image,” as used interchangeably herein, generally can refer to at least one CTA image which is a computed tomography angiography image that provides detailed visualization of blood vessels and tissues within the body. These terms may encompass both individual slices obtained during the scanning process and the reconstructed images that may represent various anatomical planes or three-dimensional views derived from the original data set. The images can be either two-dimensional (2D) or three-dimensional (3D). The images may be presented in various formats, including binary (black and white), grayscale, colored, or enhanced with contrast.

The present disclosure provides methods, techniques, systems, and devices configured for an automated diagnosis and analysis of vascular conditions using advanced imaging technologies and machine learning algorithms. In some embodiments, the vascular conditions can include peripheral vascular disease (PVD). In some embodiments, methods, techniques, systems, and/or devices provided herein may be useful in the identification, diagnosis, and/or treatment of PVD, which encompasses a range of disorders affecting blood vessels outside of the heart and brain. PAD is a form of PVD that occurs when narrowed arteries reduce blood flow to the limbs, often leading to pain and mobility issues. In some embodiments, the vascular conditions can include peripheral artery disease (PAD). In some embodiments, the PVD diagnosed and analyzed by a method, technique, system, and/or device provided herein is PAD. In some embodiments, methods, techniques, systems, and/or devices provided herein may be useful in the identification, diagnosis, and/or treatment of PAD. Thus, in some embodiments, a method, technique, system, and/or device provided herein may allow for the quantitative detection, analysis, and characterization of lesions associated with PAD in medical images. Additionally, in some embodiments, a method, technique, system, and/or device provided herein also may reduce the time associated with analysis of the medical images to identify and characterize the presence and/or severity of PAD.

In some embodiments, a method can include using at least one computer processor. The at least one computer processor can include a processing unit configured to execute machine learning algorithms for medical image analysis, a storage unit for storing raw and processed image data, a memory unit for temporarily holding data during computation, and a communication interface for transmitting processed results to external systems such as electronic health records (EHR) or picture archiving and communication systems. The processing unit may include specialized hardware such as graphic processing units (GPUs), central processing units (CPUs), tensor processing units (TPUs), or field-programmable gate arrays (FPGAs) to accelerate deep learning computations, while the storage unit can include local or cloud-based databases. In some embodiments, the machine learning algorithms can include decision trees, support vector machines, k-nearest neighbors, and/or neural networks. The neural networks may include deep learning algorithms, where deep learning algorithms may include Vision Transformer (ViT). The machine learning can include supervised learning, unsupervised learning, and/or reinforcement learning.

In some examples, hardware implementation of the ML models can be defined by heterogeneous computing architectures that balance raw throughput with low-latency response. For deep learning models like Vision Transformers (ViT), the processing units can utilize high-throughput tensor units such as NVIDIA Tensor Cores or Google's TPU v6 architecture to handle the massive matrix-vector multiplications for attention mechanisms. In some examples, to prevent a scenario where the processor outpaces data delivery, some implementations can employ High Bandwidth Memory (HBM3e) and interconnects like NVLink, which allow for high-speed data transfer between multiple GPUs. In some implementations, the ML models can be implemented in Edge-AI in which specialized Neural Processing Units (NPUs) are integrated into imaging modalities (e.g., CT scanners or ultrasound probes). This on-device processing can minimize the reliance on transmitting large raw datasets to a central server, ensuring real-time diagnostic feedback even in bandwidth-constrained environments like mobile clinics or emergency departments.

In some examples, the implementation of the ML models can rely on a multi-layered stack designed for both performance and regulatory compliance. Frameworks such as PyTorch or TensorFlow can provide the environment for algorithm execution, often optimized for the underlying hardware using libraries like NVIDIA TensorRT or Intel OpenVINO. To manage the lifecycle of these medical models, implementations can employ MLOps (Machine Learning Operations) pipelines. These pipelines can automate the ingestion of raw data from the storage unit, perform image normalization, and handle Continuous Training (CT) to adapt models to new clinical data without manual intervention. For communication, the system can serve as a bridge between the AI model and clinical workflows by wrapping processed results in DICOM (Digital Imaging and Communications in Medicine) structured reports and HL7 FHIR (Fast Healthcare Interoperability Resources) bundles. This ensures that the communication interface can seamlessly inject AI insights directly into a physician's existing viewer, complete with Explainable AI (XAI) overlays that highlight exactly which pixels influenced the neural network's decision.

In some embodiments, the method can further include obtaining at least one computed tomography angiography (CTA) image of at least a portion of the patient. At least one CTA image may be obtained through a non-invasive imaging procedure that may utilize contrast agents to enhance the visibility of blood vessels. In some embodiments, the at least one CTA image may be obtained without using contrast agents. Some non-limiting examples of the portion of the patient that may be imaged can include areas of the patient's body that include the abdominal aorta, common iliac arteries, femoral arteries, or popliteal arteries, depending on the clinical indication for assessing peripheral artery disease (PAD). The imaging process may include the patient being positioned in a CT scanner, where multiple cross-sectional images of the patients' body are captured to create a detailed view of the vascular system, allowing for the identification of any abnormalities such as stenosis or occlusions.

In some embodiments, the method can further include providing the at least one CTA image of at least a portion of the patient as input to a machine learning model. The CTA image can be provided to the model through a secure data transfer protocol, such as a direct upload from a medical imaging device or through a cloud-based storage system that ensures compliance with data privacy regulations. The machine learning model is a trained model that has been developed using a database comprising a plurality of reference images with known characteristics associated with various vascular conditions, including but not limited to peripheral artery disease (PAD). Training the machine learning model can involve supervised learning techniques, where the model is trained on labeled datasets that include annotated images indicating the presence of lesions, stenosis, or other vascular abnormalities. Known characteristics associated with PAD that the model may learn to identify include a degree of stenosis (e.g., percentage narrowing of the artery), a presence of calcified or soft plaques, and an overall morphology of the vascular structures. The model may also be trained to recognize specific patterns indicative of PAD, such as changes in vessel diameter or irregularities in arterial walls. Additionally, the model can be continuously improved through iterative training processes, where new data is incorporated to refine its predictive capabilities. This ongoing training can enhance the model's accuracy in diagnosing PAD and other vascular conditions, ultimately supporting clinical decision-making.

In some embodiments, the method can include classifying characteristics of at least a portion of the vascular system based on the at least one CTA image of at least a portion of the patient using the trained machine learning model. The trained machine learning model can be configured to analyze the CTA image and identify various features of the vascular system, including, but not limited to, the presence of lesions, degree of stenosis, and morphological changes indicative of peripheral artery disease (PAD). The model can classify these characteristics by recognizing patterns and anomalies within the image data, allowing for a detailed assessment of the vascular condition. This classification process may involve the extraction of quantitative metrics, such as the percentage of narrowing in affected arteries, as well as qualitative assessments of the overall vascular health.

In some embodiments, determining a diagnosis of the patient can be based on the classified characteristics of at least a portion of the vascular system from the at least one CTA image of at least a portion of the patient, where the diagnosis can include at least one condition of the patient correlated to at least one indicator of peripheral artery disease (PAD). The trained machine learning model may analyze the classified characteristics to determine the presence and severity of vascular abnormalities, facilitating an accurate diagnosis. The process may involve comparing the identified characteristics against established criteria for PAD, allowing clinicians to make informed decisions regarding the patient's condition.

In some embodiments, generating one or more groups of images from the at least one CTA image can be based on characteristics associated with peripheral artery disease (PAD), where the generated one or more groups of images can include segmented images highlighting vascular abnormalities, annotated images, and comparative images. The process may involve utilizing the trained machine learning model to analyze the CTA image and identify specific features indicative of PAD. Segmented images may focus on delineating areas of interest, such as lesions or stenosis, while annotated images can provide additional context or information regarding the identified abnormalities. Comparative images may be generated to contrast the patient's vascular structures with reference images, aiding in the assessment of the severity and nature of the condition.

In some embodiments, matching images from the one or more groups of images to at least one reference image with known characteristics associated with peripheral artery disease (PAD) can be performed, where the matching process is conducted by the trained machine learning model. The model can analyze the features of the images generated from the CTA data and compare them to the reference images to identify similarities and differences. The matching process may involve evaluating various characteristics, such as lesion morphology, degree of stenosis, and other relevant features indicative of PAD.

In some embodiments, determining a diagnosis of the patient can be based on the generated one or more groups of images, where the diagnosis can include at least one condition of the patient correlated to at least one indicator of peripheral artery disease (PAD). The trained machine learning model can analyze the characteristics of the images, such as the presence of lesions, the degree of stenosis, and other vascular abnormalities, to determine the likelihood of PAD. The analysis may involve comparing the identified features against established diagnostic criteria and thresholds for PAD, allowing for a more accurate and reliable diagnosis. In some embodiments, following the confirmation of the diagnosis, generating a report based on the diagnosis can occur, where the report can include a diagnosis of PAD, an assessment of the severity of PAD, and recommendations for treatment options. The report may detail the specific findings from the imaging analysis, including the percentage of stenosis, the location and morphology of lesions, and any other relevant vascular characteristics. Additionally, the report can provide treatment recommendations tailored to the patient's condition, which may include options such as lifestyle modifications, medication, or interventional procedures. This comprehensive report can serve as a valuable tool for clinicians in making informed decisions regarding patient management and care. In some embodiments, following the confirmation of the diagnosis of PAD, generating a 2D/3D vascular map can occur, where the vascular map provides visualization data that may be used, for example, as an input for a 3D printing tool to generate a 3D printed vascular map. The 2D/3D vascular map may be a valuable tool for clinicians in preparing and conducting interventions, including, but not limited to, surgery or device implantation.

In some embodiments, the characteristics associated with peripheral artery disease (PAD) may be related to a blockage in a vessel, where the cross-sectional area of the blockage can be at least about 0%, at least about 5%, at least about 10%, at least about 15%, at least about 20%, at least about 25%, at least about 30%, at least about 35%, at least about 40%, at least about 45%, at least about 50%, at least about 55%, at least about 60%, at least about 65%, at least about 70%, at least about 75%, at least about 80%, at least about 85%, at least about 90%, at least about 95%, or up to at least about 100% of the cross-sectional area of the vessel. This threshold can serve as an indicator for assessing the severity of the vascular condition. The trained machine learning model can analyze the computed tomography angiography (CTA) images to accurately quantify the extent of the blockage, providing metrics that facilitate diagnosis and inform treatment planning for the patient.

The performance of AI-generated segmentation results can be evaluated using a Dice metric (i.e., Dice), also known as the Dice coefficient or Dice similarity coefficient (DSC). The Dice as a statistical measure can be employed to assess the similarity between a segmented image produced by the trained machine learning model and a segmented image created by expert radiologists. In some embodiments, the Dice may be utilized to quantify the accuracy of the machine learning model in classifying elements within a CTA image. The Dice can be in a range of 0% to 100%. A Dice score of 0% indicates no overlap between the segmented image produced by the machine learning model and the segmented image created by expert radiologists. This means that the model's segmentation is completely inaccurate and fails to capture any relevant structures as defined by the experts. A Dice score of 100% indicates perfect overlap between the two segmented images. This means that the model has accurately identified and segmented the structures exactly as the expert radiologists have defined them. A higher Dice score indicates greater accuracy in segmentation. In some embodiments, the Dice can be about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. In some embodiments, the Dice can be about 53%, about 62%, about 84%, about 87%, about 91%, or about 92%.

The Dice can be calculated using the formula:

$$Dice coefficient = 2 * \vert A ⋂ B \vert / ( \vert A \vert + \vert B \vert )$$

where (A) represents the set of pixels in the AI-generated segmentation, and (B) represents the set of pixels in the expert-drawn segmentation. A Dice coefficient value of 1 indicates perfect overlap, while a value of 0 indicates no overlap. By utilizing the Dice metric, the accuracy and reliability of the model's segmentation performance can be assessed.

In some embodiments, sensitivity and specificity can be used to evaluate the performance of the model. The sensitivity measures the proportion of actual positive cases (e.g., correctly identified segments) that are correctly identified by the model. It reflects the model's ability to detect relevant structures. A high sensitivity indicates that the model is effective at identifying positive cases, minimizing false negatives. In some embodiments, the sensitivity can be ranged between about 5% to about 100%. In some embodiments, the sensitivity can be about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%. The specificity measures the proportion of actual negative cases (e.g., correctly identified non-segmented areas) that are correctly identified by the model. It reflects the model's ability to avoid false positives. A high specificity indicates that the model is effective at correctly identifying negative cases, minimizing false positives. In some embodiments, the specificity can be ranged between about 5% to about 100%. In some embodiments, the specificity can be about 0%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100%.

In some embodiments, a loss function can be used to quantify the difference between a predicted output of a model and an actual target value. The loss function can be used during the training process of the machine learning model, guiding an optimization of the machine learning model's parameters to improve its predictive accuracy. In the context of classifying characteristics associated with peripheral artery disease (PAD), the loss function can minimize classification errors by adjusting the model's weights based on discrepancies between predicted and true classifications. In some embodiments, the true classifications can be referred to a classification conducted by an expert or a radiologist. In other embodiments, the true classifications can be referred to a classification conducted by the machine learning model and labeled as a true classification. In some embodiments, cross-entropy loss as type of loss function, can be used for measuring the performance of the machine learning model. A binary cross-entropy loss can be ranged between 0 and 1.0. In some embodiments, the binary cross-entropy loss can be about 0, about 0.1, about 0.15, about 0.2, about 0.25, about 0.3, about 0.35, about 0.4, about 0.45, about 0.5, about 0.55, about 0.6, about 0.65, about 0.7, about 0.75, about 0.8, about 0.85, about 0.9, about 0.95, or about 1.0. Cross-entropy loss can calculate a dissimilarity between a predicted probability distribution and an actual distribution of the target classes. In some embodiments, a cross-entropy loss value below 0.1 may indicate strong model performance, suggesting that the machine learning model's classifications are closely aligned with the true classifications, thereby reflecting high confidence in its outputs. In some embodiments, categorical cross-entropy, mean squared error (MSE), mean absolute error (MAE), hinge loss, and/or focal loss can be used in classifying characteristics associated with PAD.

In some embodiments, obtaining the at least one CTA image can include receiving at least one Digital Imaging and Communications in Medicine (DICOM) image, where the DICOM image can be associated with the patient having peripheral artery disease (PAD). The DICOM image can contain metadata, including patient identification, imaging parameters, and/or acquisition details. The metadata can include Patient ID, which can be a unique identifier assigned to a patient; Patient Name, which can be the full name of the patient; Date of Birth, which can be the patient's date of birth used for age calculation; Gender, which can indicate the patient's gender; Study Instance UID, which can be a unique identifier for the imaging study; Study Date and Time, which can represent when an imaging study was performed; and Referring Physician, which can be the name of a physician who requested the imaging study. The metadata may include Imaging Modality, which can specify the type of imaging technique used (e.g., CTA, MRI, ultrasound), and Acquisition Parameters, which can detail settings and protocols used during the imaging process. The metadata may include Slice Thickness, which is the thickness of each slice in the imaging study and can affect the resolution and quality of the images; Image Dimensions, which refer to the size of the image in pixels (width and height); Pixel Spacing, the physical distance between the centers of adjacent pixels; Bits Allocated, the number of bits used to represent each pixel, affecting image quality and grayscale levels; Clinical History, which includes relevant medical history or notes that may assist in interpreting the images; Diagnosis, any preliminary diagnosis or findings related to the imaging study; Image Orientation, which provides information about how the image is oriented in relation to the patient's anatomy; Positioning Information, detailing the patient's position during the imaging procedure (e.g., supine, prone); and Contrast Agent Information, detailing any contrast agents used during the imaging procedure. The system can process the DICOM image to extract relevant information and prepare it for input into the trained machine learning model.

In some embodiments, the at least one associated tag may be coupled with at least one image among the at least one DICOM image, generating at least one tagged DICOM image. The tagging process can involve associating relevant metadata with the DICOM image, such as patient information, imaging parameters, and diagnostic findings. This tagged DICOM image can facilitate easier retrieval and analysis of the imaging data, allowing for more efficient processing by the trained machine learning model. The inclusion of associated tags can enhance the organization of the imaging dataset and improve the accuracy of subsequent analyses, as the model can leverage this additional context when identifying and classifying vascular conditions. In some embodiments, the at least one DICOM image may be organized into coherent series by the machine learning model. The organization process can involve analyzing the DICOM images to identify their spatial relationships and sequence, ensuring that the images are arranged in a manner that accurately reflects the anatomical structure being examined. This coherent organization can facilitate more effective analysis and interpretation of the imaging data, allowing the trained machine learning model to better identify and classify vascular conditions. By structuring the images into series, the model can enhance its ability to detect abnormalities, assess the progression of disease, and support clinical decision-making.

In some embodiments, the machine learning model may be configured to create a structured database of the at least one tagged DICOM image. This structured database can facilitate the organization and retrieval of imaging data, allowing for efficient access to relevant information associated with each tagged image. The model can categorize the tagged DICOM images based on various criteria, such as patient demographics, imaging parameters, and diagnostic findings. By maintaining a structured database, the system can enhance the ability to perform large-scale analyses, support longitudinal studies, and improve the overall workflow for healthcare providers. Additionally, this organization can aid in the training of the machine learning model by providing a well-defined dataset for further learning and refinement. In some embodiments, the machine learning model may be configured to create a primary classification of the at least one tagged DICOM image. This primary classification can involve analyzing the features and characteristics of the tagged DICOM image to categorize it based on predefined criteria associated with various vascular conditions. The model can utilize learned patterns from the training dataset to identify attributes, such as the presence of lesions, the degree of stenosis, or other relevant abnormalities. By establishing a primary classification, the model can streamline the diagnostic process, enabling healthcare providers to quickly assess the patient's condition and determine appropriate next steps for treatment or further evaluation.

In some embodiments, the primary classification of the at least one tagged DICOM image may include an identification of axial series. This classification process can involve analyzing the tagged DICOM images to determine their orientation and alignment within the axial plane, which is useful for improved accuracy of anatomical representation. The machine learning model can utilize learned features to recognize patterns indicative of axial series, allowing for the systematic organization of images based on their spatial relationships. By identifying axial series, the model can enhance the efficiency of subsequent analyses, enabling more precise assessments of vascular conditions and facilitating improved diagnostic accuracy. In some embodiments, the identification of axial series may include evaluating the thickness of a slice of the at least one image among the at least one DICOM image. This evaluation can involve analyzing the pixel dimensions and spatial resolution of the DICOM images to determine the appropriate slice thickness for accurate anatomical representation. By assessing the thickness, the machine learning model can ensure that the images are correctly aligned and organized into axial series, which is useful for comprehensive analysis of the vascular structures. This process can enhance the model's ability to detect and classify vascular abnormalities, providing clinicians with precise information for diagnosis and treatment planning.

In some embodiments, the identification of axial series may include assessing anatomical coverage. This assessment can involve evaluating the extent to which the DICOM images capture relevant anatomical structures within the vascular system. By determining the anatomical coverage, the machine learning model can ensure that the axial series includes regions that can aid in comprehensive analysis. This process can accurately identify and classify vascular conditions, as it allows the model to focus on the entirety of the affected area, thereby enhancing diagnostic accuracy and supporting effective treatment planning. In some embodiments, the identification of axial series may include evaluating quality metrics of the at least one tagged DICOM image. This evaluation can involve assessing various quality indicators, such as signal-to-noise ratio, contrast resolution, and artifacts present in the images. By analyzing these quality metrics, the machine learning model can determine the suitability of the tagged DICOM images for accurate interpretation and analysis. Ensuring high-quality images is useful for the effective identification and classification of vascular conditions, as it directly impacts the model's performance and the reliability of the diagnostic outcomes. This process can facilitate the generation of coherent axial series that enhance the overall diagnostic workflow.

In some embodiments, the trained machine learning model may be configured to automatically identify and label anatomical structures, where the anatomical structures can include the aorta, common iliac arteries, external right and left iliac arteries, left common femoral arteries, right common femoral arteries, left superficial femoral arteries, right superficial femoral arteries, left popliteal arteries, right popliteal arteries, left anterior tibial artery (ATA) bifurcation, and right ATA bifurcation. The model may utilize convolutional neural networks (CNNs) to analyze the spatial patterns in the imaging data, enabling it to recognize specific features associated with each anatomical structure. It can also employ image segmentation techniques to delineate boundaries of structures such as the aorta and femoral arteries, allowing for precise labeling. Additionally, the model may incorporate transfer learning, leveraging pre-trained models on similar tasks to enhance its accuracy in identifying vascular anatomy. The use of data augmentation techniques can further improve the model's robustness by exposing it to a wider variety of imaging conditions. Lastly, the model can analyze the metadata associated with the DICOM images to assist in determining the identity of the anatomical structures based on their expected locations and relationships within the vascular system. The automatic identification process can enhance the efficiency of image analysis by reducing the reliance on manual annotation by radiologists. By accurately labeling these structures, the model can facilitate more precise assessments of vascular conditions, support the generation of comprehensive reports, and ultimately improve clinical decision-making regarding patient care.

In some embodiments, the trained machine learning model may be configured to optimize a processing range for the automatic identification and labeling of anatomical structures. This optimization can involve adjusting the model's parameters to enhance its sensitivity and specificity in detecting various structures within the vascular system. The model can utilize techniques such as hyperparameter tuning to find the most effective settings for processing the imaging data, thereby improving accuracy in identifying structures like the aorta, iliac arteries, and femoral arteries. Additionally, the model may implement adaptive learning strategies that allow it to refine its processing range based on feedback from previous analyses, ensuring that it remains effective across different imaging modalities and patient anatomies. By optimizing the processing range, the model can enhance its performance, leading to more reliable and efficient labeling of anatomical structures in medical imaging studies. In some embodiments, the trained machine learning model may be configured to identify medical devices within at least a portion of the vascular system associated with the at least one CTA image of the at least a portion of the patient, where the medical devices can include stents or bypass grafts. The model can analyze the CTA images to detect the presence and location of these devices, utilizing pattern recognition algorithms to differentiate between the anatomical structures and the implanted devices. By leveraging features such as shape, size, and radiopacity, the model can accurately classify the devices and assess their integration within the vascular system.

In some embodiments, the trained machine learning model may be configured to identify imaging artifacts within at least a portion of the vascular system associated with the at least one CTA image of the at least a portion of the patient, where the imaging artifacts can include motion artifacts, streak artifacts, or beam hardening artifacts. The model can analyze the CTA images to detect these artifacts by recognizing patterns that deviate from expected anatomical structures. For instance, it may utilize advanced filtering techniques to differentiate between true vascular features and artifacts caused by patient movement during imaging. Additionally, the model can employ machine learning algorithms trained on annotated datasets containing examples of various artifacts, enabling it to accurately classify and label the types of artifacts present in the images. To identify motion artifacts, the model can analyze the temporal consistency of pixel values across multiple slices or frames. By employing techniques such as optical flow analysis, the model can detect discrepancies in the expected movement of anatomical structures, indicating that the patient may have moved during the imaging process. Additionally, the model can utilize recurrent neural networks (RNNs) to capture temporal dependencies in the imaging data, enhancing its ability to recognize patterns indicative of motion artifacts. For streak artifacts, which often appear as bright lines or bands across the image, the model can apply edge detection algorithms to identify abnormal linear features that do not correspond to anatomical structures. Techniques such as convolutional neural networks (CNNs) can be trained on labeled datasets containing examples of streak artifacts, enabling the model to learn to distinguish these artifacts from true vascular features. In the case of beam hardening artifacts, which occur due to the differential absorption of X-rays by dense structures, the model can analyze the intensity profiles of the images. By employing histogram analysis and machine learning techniques, the model can identify areas where the attenuation of X-rays leads to dark bands or streaks. The model may also incorporate physical modeling of X-ray interactions to better understand and predict where beam hardening is likely to occur.

In some embodiments, the method can include digitally segmenting regions within at least one image of at least a portion of the vascular system associated with the at least one CTA image of the at least a portion of the patient, where the segmented images can include arterial walls, lesions, or thrombosis. The trained machine learning model may utilize image segmentation algorithms, such as U-Net or Mask R-CNN, to accurately delineate the boundaries of these structures. By analyzing pixel intensity and spatial relationships, the model can differentiate between healthy tissue and pathological features. This segmentation process allows for a detailed assessment of the vascular condition, enabling clinicians to visualize the extent and location of lesions or thrombosis. In some embodiments, the method can include calculating an area ratio and/or percentage associated with stenosis using the trained machine learning model. The model may analyze the segmented images to quantify the cross-sectional area of the vessel and the area of any identified lesions. By comparing these areas, the model can compute the percentage of stenosis, providing a clear metric for assessing the severity of vascular occlusion. This quantitative analysis can enhance the diagnostic process by offering precise measurements that support clinical decision-making regarding interventions or further evaluations. In some embodiments, the method can include classifying a lesion's morphology using the trained machine learning model, where the lesion is identified within at least a portion of the vascular system associated with the at least one CTA image of the at least a portion of the patient. The model may utilize feature extraction techniques to analyze the shape, size, and texture of the lesions, categorizing them into predefined morphological classifications such as calcified, soft, or mixed lesions. By leveraging a dataset of annotated images, the model can learn to recognize specific morphological patterns associated with different types of lesions.

In some embodiments, post-processing steps can be employed to refine, enhance or clean up the images before the classification module begins. The post-processing steps may include noise reduction techniques, such as Gaussian filtering or median filtering, which can help eliminate artifacts and improve image quality, for example by smoothing pixel intensity variations. In some embodiments, morphological operations, such as dilation and erosion, can be applied to refine the segmented regions, ensuring that vessel structures are well-defined and free from irrelevant components. In some embodiments, connected component analysis may assist in identifying and removing isolated regions that do not correspond to actual vessel structures. In some embodiments, thresholding techniques such as Global Thresholding, Adaptive Thresholding, Multi-level Thresholding, Binarization with Histogram Analysis, K-means Clustering, Canny Edge Detection, IsoData Thresholding, Otsu's Method, Deep Learning-based Segmentation (e.g., U-Net, Mask R-CNN), Fuzzy C-means Clustering, or Thresholding with Convolutional Neural Networks (CNNs) can be utilized to segment, enhance contrast and delineate vessel boundaries. In some embodiments, tracking can also be integrated as a post-processing step, employing methods such as Kalman filters or particle filters to monitor the movement and evolution of vessel segments over time. The tracking process may involve associating vessel segments across frames or slices, ensuring continuity in the analysis.

In some embodiments, the tracking process can be integrated into the processing of 2D segmentation masks generated by a deep learning-based semantic segmentation algorithm, which identifies different tissues such as lumen, lesions, or thrombosis. The input images may contain one or two arteries of interest, resulting in output lumen masks that may include connected components. In some embodiments, a proposed algorithm can process the generated 2D lumen segmentation masks to remove false positive regions that do not correspond to the actual lumen using automated techniques such as a graph-based approach. The process may involve applying a connected component (CC) labeling algorithm to each 2D slice to generate CCs, which can be represented as nodes in a graph. Edges can be created between CCs of adjacent slices based on proximity and similarity, with weights defined by techniques such as a combination of Euclidean distance and Dice scores. In some embodiments, a graph traversal algorithm, such as the minimum spanning tree (MST), can be utilized to connect all nodes with minimal added weight. Post-processing steps may include the removal of orphaned nodes, which are identified as those connected to only one other node, In some embodiments, an algorithm such as Dijkstra's Algorithm can be employed to find the shortest weighted path, applied separately for slices before and after bifurcation.

In some embodiments, the method can include providing treatment guidance based on the classification of the lesion's morphology using the trained machine learning model, where the treatment guidance can be selected from a set of recorded treatments according to the classification of the lesion's morphology. The model may analyze the morphological characteristics of the lesion, such as its size, shape, and composition, to determine the most appropriate treatment options. This guidance can be derived from a database of recorded treatments that have been approved by certified clinicians, ensuring that the recommendations are clinically relevant and evidence-based. By offering tailored treatment options, the model can assist healthcare providers in making informed decisions regarding patient management. In some embodiments, the method can include providing guidance on selecting an intervention device based on the classification of the lesion's morphology and/or lesion characteristics using the trained machine learning model. The guidance on selecting the intervention device can be selected from a set of recorded guidance according to the classification of the lesion's morphology and/or the lesion's characteristics, where the recorded guidance can be approved by certified clinicians. The model may evaluate the specific features of the lesion, such as its location and type, to recommend suitable intervention devices, such as stents or balloon catheters. The identification of a treatment option can enhance the precision of interventional procedures and improve patient outcomes by ensuring that the most appropriate devices are utilized for each unique case. In some embodiments, the method can include generating a structured radiological report using the trained machine learning model, where the report can include at least one condition of the patient correlated to at least one indicator of peripheral artery disease (PAD). The structured report may include detailed findings from the imaging analysis, such as the presence and severity of lesions, measurements of stenosis, and any relevant anatomical landmarks.

In some embodiments, the method can include generating a structured radiological report using the trained machine learning model, where the report can include at least one lesion's morphology, anatomical landmarks, and the spatial distribution of the at least one lesion within the vascular system. The model may extract and compile relevant data from the imaging analysis, presenting it in a structured format that enhances clarity and usability for clinicians. This report can detail the characteristics of the lesions, including their size, shape, and type, as well as the specific anatomical landmarks that provide context for their location. In some embodiments, the method can include generating a structured radiological report that includes a 3D vascular mapping, detailed lesion characterization, quantitative measurements, and/or treatment recommendations. The 3D vascular mapping can provide a comprehensive visualization of the vascular structures, allowing clinicians to better understand the spatial relationships between lesions and surrounding anatomy. Detailed lesion characterization may include descriptions of the lesion's morphology and any associated abnormalities. Quantitative measurements can offer precise metrics, such as the percentage of stenosis, while treatment recommendations can be tailored based on the findings, guiding clinicians in selecting appropriate interventions.

In some embodiments, the method can include generating a comprehensive structured radiological report using the trained machine learning model. The report may include a variety of formats, such as text, tables, graphs, videos, and 2D or 3D graphical mapping or visualization of results. In some embodiments, the report may contain a textual summary of the patient's condition correlated with indicators of peripheral artery disease (PAD), accompanied by tables that present quantitative measurements, such as the percentage of stenosis across different vascular segments. Graphs may be included to illustrate trends over time, such as changes in lesion size or severity, providing a visual representation of disease progression. In some embodiments, the mapped and/or visualized results may be analyzed alongside other results, such as text results, using the same computer application. For example, a clinician can use an integrated software platform that allows them to view a 3D model of the vascular system alongside a textual report detailing the patient's medical history and imaging findings. In other embodiments, the mapped and/or visualized results may be analyzed with other results using different computer applications. For instance, a clinician may generate a 2D/3D vascular map using one application and then export the data to a separate analytics tool to visualize 2D/3D results or to generate a statistical analysis.

In some embodiments, the method can include generating a 2D/3D vascular map. In some embodiments, a visualization tool may be employed to generate the 2D/3D vascular map. For example, a virtual reality (VR) application can be used to generate a 3D reconstruction of the vascular anatomy. In some embodiments, an augmented reality (AR) tool can overlay imaging data onto a physical model of the vascular system, allowing for real-time interaction and analysis during clinical assessments. In some embodiments, the model can provide visualization data, including, but not limited to, a comprehensive visualization of the vascular structures, that may be used as an input for a 3D printing tool to generate a 3D printed vascular map. The model can extract and compile relevant data from the imaging analysis, presenting it in a 3D vascular map that can enhance usability for clinicians in downstream applications, including, but not limited to, allowing clinicians to better understand the spatial relationships between lesions and surrounding anatomy and planning surgical interventions to treat identified lesions.

In some embodiments, the method can include utilizing a neural network model as the trained machine learning model. This neural network model can be designed to process complex imaging data and learn from large datasets, enabling it to identify and classify various vascular conditions effectively. The architecture of the neural network can be optimized for performance, allowing it to handle the intricacies of medical imaging analysis. In some embodiments, the neural network model can include one or more single-head U-Net (SHU) and/or multi-head U-Net (MHU) convolutional layers. This architecture is designed with an encoder-decoder structure, where the encoder progressively downsamples the input image to extract high-level features, while the decoder upsamples the feature maps to produce a segmentation mask that aligns with the original image dimensions. The single-head U-Net can focus on specific tasks, such as segmenting a particular anatomical structure or identifying a specific type of lesion, making it effective for targeted analyses. In some embodiments, the multi-head U-Net can facilitate simultaneous processing of multiple outputs, enhancing the model's ability to analyze various aspects of the vascular system. For instance, it can generate separate segmentation masks for arteries, veins, and lesions in a single forward pass, improving efficiency and reducing the computational burden. Additionally, the multi-head architecture allows for the integration of different types of information, such as morphological characteristics and functional assessments, enabling a more comprehensive evaluation of the vascular condition. By leveraging the strengths of the U-Net architecture, the model can achieve high accuracy in segmenting complex vascular structures, ultimately supporting improved diagnostic and treatment planning capabilities. In some embodiments, the characteristics of lesions can include hard lesions, soft tissue lesions, or mixed lesions. The model may be trained to recognize these different types of lesions based on their features, such as density, texture, and morphology. This classification can aid in determining the appropriate treatment strategies and assessing the severity of the vascular condition. In some embodiments, the segmented images can include a segment correlated with a blockage in a vessel within at least a portion of the vascular system. The model can analyze the segmented images to identify specific areas where blockages occur. In some embodiments, the blockage in the vessel can include hard lesions, soft tissue lesions, or mixed lesions. The model may differentiate between these types of blockages based on their characteristics, allowing for tailored treatment recommendations and improved patient management. In some embodiments, the trained machine learning model can be configured to identify arteries within the vascular system based on an analysis of at least one CTA image. This identification process may involve segmenting the arterial structures and classifying them according to their anatomical features, enhancing the model's ability to provide accurate assessments of vascular health.

In some embodiments, the method can include configuring the trained machine learning model to distinguish between arteries and veins within the vascular system based on an analysis of at least one CTA image. The model may utilize feature extraction techniques to analyze the intensity, shape, and spatial relationships of the vascular structures in the CTA image. By training on a labeled dataset that includes examples of both arteries and veins, the model can learn to identify distinguishing characteristics, enabling accurate classification of these structures. In some embodiments, the method can include configuring the trained machine learning model to isolate at least one artery from a plurality of arteries identified within the vascular system based on an analysis of at least one CTA image. The model may employ segmentation algorithms to delineate the boundaries of the arteries, allowing for precise isolation of the target artery. This capability can facilitate focused analysis and reporting on specific vascular segments, which is useful for diagnosing conditions such as stenosis or occlusion. In some embodiments, the method can include configuring the trained machine learning model to generate a histogram representing the characteristics of lesions. The histogram can provide a visual representation of various metrics, such as the size, shape, and density of the lesions identified in the imaging data. By analyzing these characteristics, clinicians can gain insights into the nature and severity of the lesions, supporting more informed decision-making regarding treatment options. In some embodiments, the method can include configuring the trained machine learning model to generate a 3D vascular map based on at least one CTA image. This 3D vascular map can provide a comprehensive visualization of the vascular structures, allowing for better assessment of the spatial relationships between arteries, veins, and lesions. The model may utilize volumetric rendering techniques to create the 3D representation, enhancing the clinician's ability to visualize complex anatomical configurations.

In some embodiments, the method can include configuring the trained machine learning model to recommend a treatment plan, where the treatment plan can include a recommended use of an interventional tool based on a 3D construction of lesions identified within the vascular system. The model may analyze the characteristics of the lesions and their spatial relationships to suggest appropriate interventional devices, such as stents or balloons, tailored to the specific needs of the patient. In some embodiments, the method can include generating a report that includes anatomical locations and/or dimensions of lesions identified within the vascular system. The report can provide detailed information on the size and location of each lesion, enhancing the clinician's understanding of the patient's vascular health. This information can be used for treatment planning and monitoring the progression of vascular conditions.

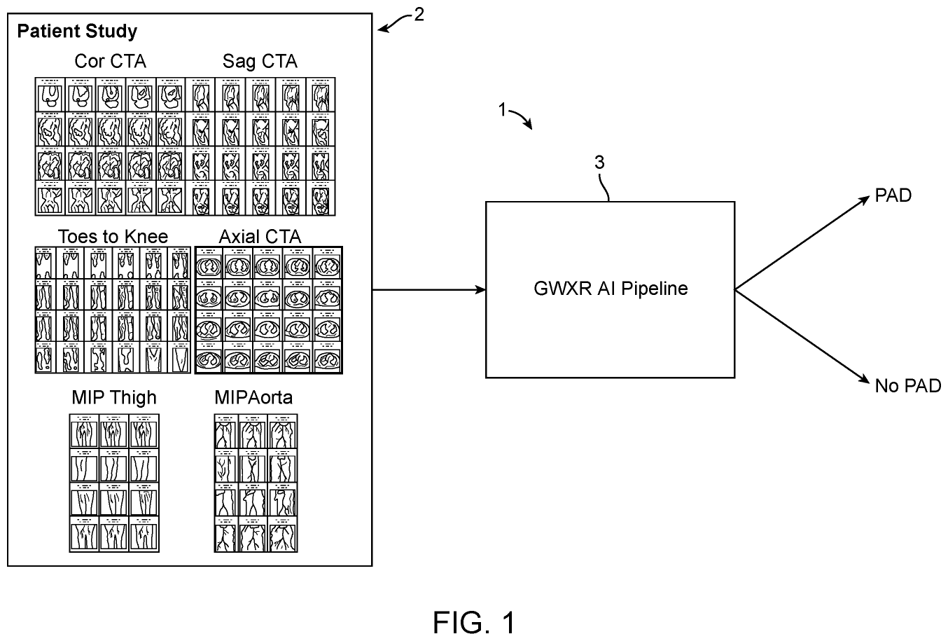

FIG. 1 illustrates an example workflow 1 for the classification of peripheral artery disease (PAD). The figure displays obtained CTA images 2, including coronal, sagittal, axial, maximum intensity projection (MIP) images of the aorta, and MIP images of the thigh CTA from a patient. These images 2 are transferred to an AI tool 3 that has been trained to detect the occurrence of PAD. The AI tool 3 analyzes the CTA images 2 and provides a report classifying the vessels as having PAD or not.

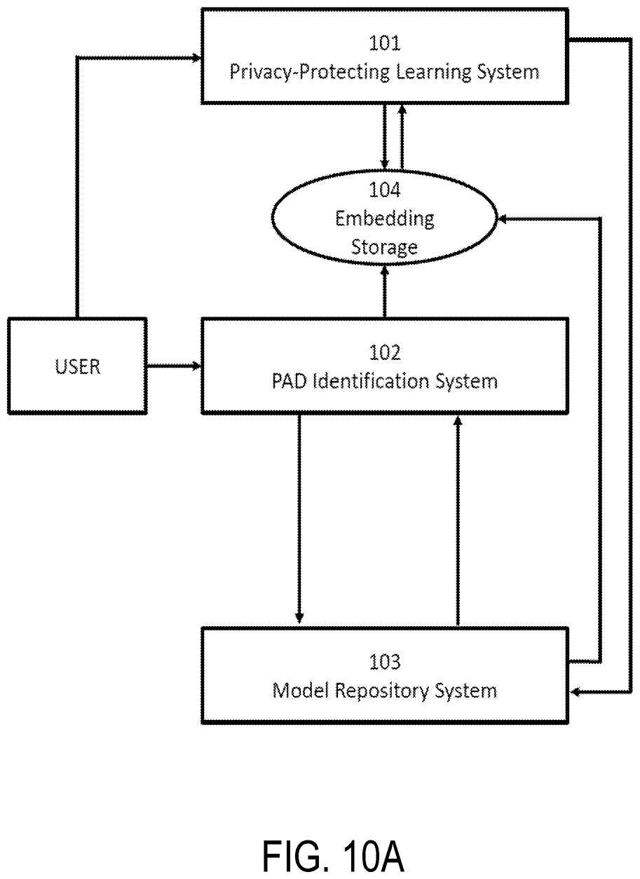

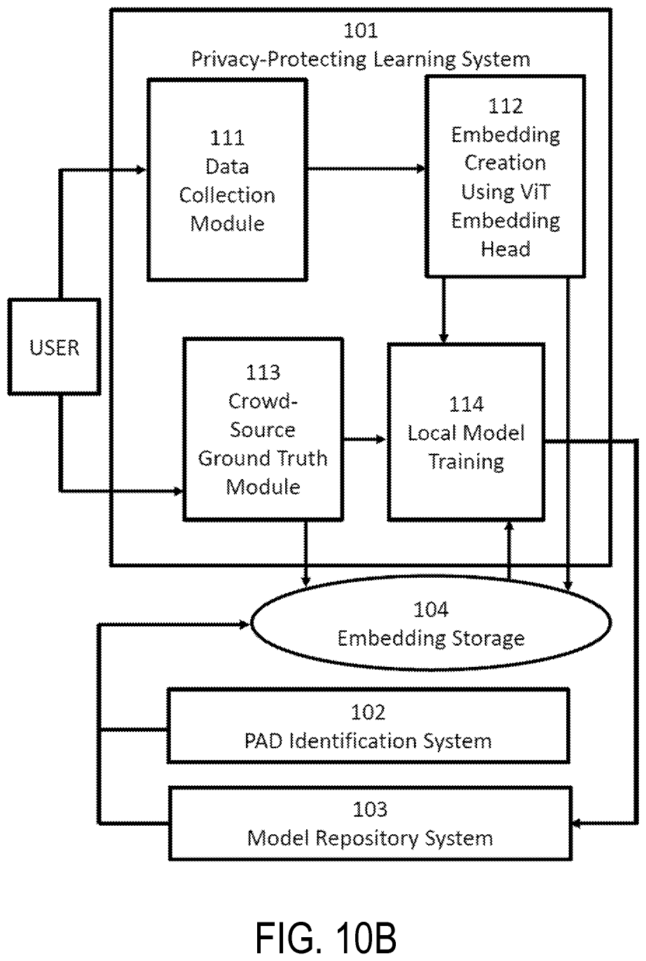

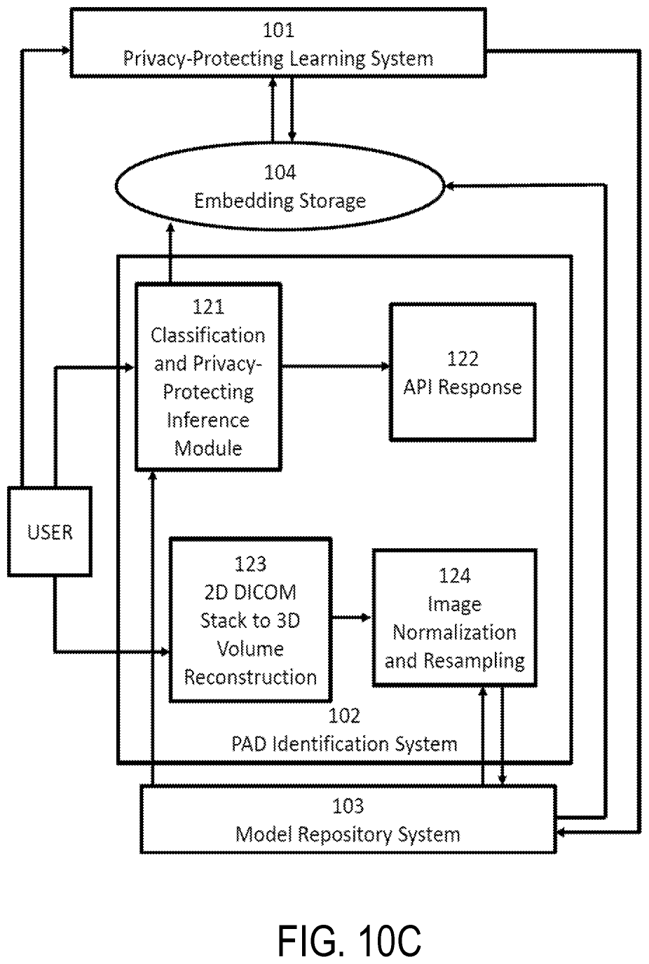

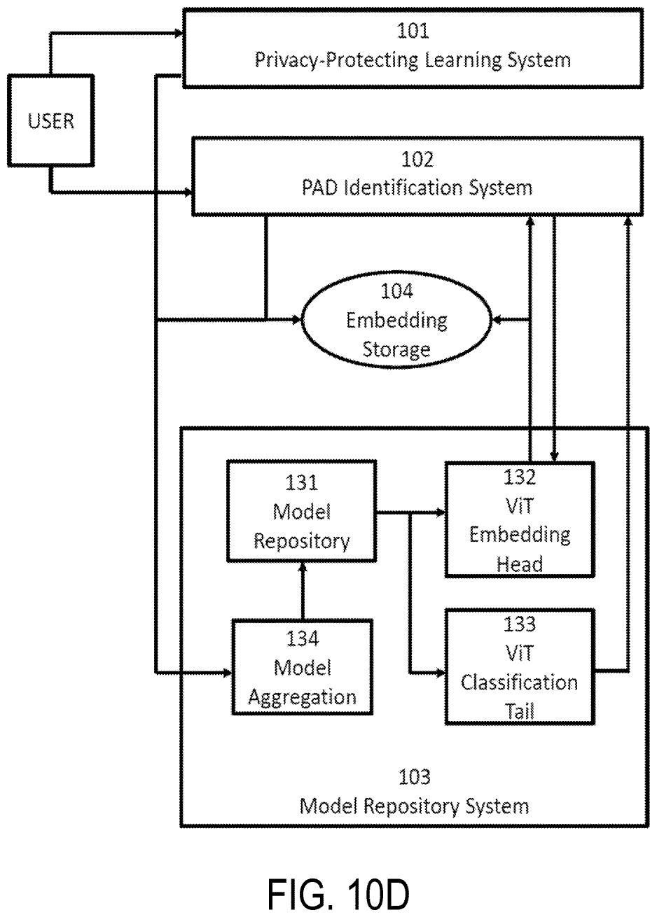

FIG. 10A illustrates an exemplary flow chart for classifying maximum intensity projections (MIPs) and 3D computed tomography angiography (CTA) volumes to identify peripheral artery disease (PAD) in patients, utilizing a privacy-protecting inference paradigm. At least one type of model introduced in this disclosure is a vision transformer (ViT model), which is designed to effectively analyze imaging data and enhance the accuracy of PAD identification while ensuring patient privacy. The system includes several interconnected modules designed to facilitate the classification of maximum intensity projections (MIPs) and 3D computed tomography angiography (CTA) volumes for identifying peripheral artery disease (PAD) in patients (see FIGS. 10B-10D). Data collected from the user are transferred to Privacy-Protecting Learning System 101 and PAD Identification System 102. The Privacy-Protecting Learning System 101 communicates with Embedding Storage 104 including send and/or receive processes. The PAD Identification System 102 also send data to Embedding Storage 104. The PAD Identification System 102 communicates with Model Repository System 103 including send and/or receive processes. The Model Repository System 103 send data to Embedding Storage 104. The Privacy-Protecting Learning System 101 send data to Model Repository System 103 as well.

As exemplified in FIG. 10B, the Privacy-Protecting Learning System 101 includes:

Data Collection Module 111: This module collects user-provided data, including images and metadata, for use in the privacy-protecting learning system, facilitating the integration of new information into the model.

Embedding Creation using ViT Embedding Head 112: This module employs the VIT Embedding Head 132 to create embeddings for the collected data, which are stored in the Embedding Storage 104 and used in local model training.

Crowd-Sourced Ground Truth Module 113: This module collects ground truth labels for the collected data from the user or other experts to ensure the accuracy of the training process.

Local Model Training 114: This module trains a local model using the embeddings and ground truth labels sourced from the Crowd-Sourced Ground Truth Module 113 and Embedding Storage 104, enhancing the model's accuracy.

As exemplified in FIG. 10C, the PAD Identification System 102 includes:

Classification and Privacy-Protecting Inference Module 121: This module utilizes the ViT Classification Tail 133 to classify the input images and generate the API Response 122, ensuring that patient data remains secure throughout the process.

2D DICOM Stack to 3D Volume Reconstruction 123: This module converts a 2D DICOM stack of images provided by the user into a 3D volume representation, enabling a comprehensive view of the vascular structures.

Image Normalization and Resampling 124: This module normalizes and resamples the 3D volume to ensure consistency in size and intensity values across the dataset for improved accuracy of analysis.

As exemplified in FIG. 10D, the Model Repository System 103 includes:

ViT Embedding Head 132: Sourced from the Model Repository 131, this component is the head of the Vision Transformer (ViT) model responsible for creating embeddings from the input images, capturing desired features for subsequent processing.

Embedding Storage 104: This module stores the generated embeddings for future use in model training and privacy-protecting inference, ensuring that valuable data is retained for ongoing improvements. The Embedding Storage 104 can provide data to the Local Model Training 114 of the Privacy-Protecting Learning System 101. The transformation of high-resolution 3D CTA volumes into dense vector embeddings represents a significant architectural optimization, primarily achieved through the mechanism of dimensionality reduction. While a standard 3D medical scan can include millions of voxels—often consuming hundreds of megabytes or even gigabytes of storage—an embedding captures the semantic and anatomical features of that scan in a compact numerical format. By distilling complex volumetric data into these low-dimensional representations, the system effectively strips away redundant information while preserving the diagnostic features desired for machine learning tasks.

This reduction in the data footprint directly alleviates bottlenecks in distributed learning and cloud-based environments. Communicating these lightweight vectors instead of raw, heavy-duty image files ensures that the system can operate within constrained bandwidth environments without compromising performance. Furthermore, the smaller payload size significantly minimizes transmission time and network congestion, leading to a marked decrease in end-to-end latency. This efficiency can be beneficial for maintaining responsive, real-time privacy-protecting inference and local model training, as it allows for rapid data synchronization and reduced computational overhead at the edge.

ViT Classification Tail 133: Also sourced from the Model Repository 131, this component is the tail of the ViT model responsible for performing classification tasks, determining the presence of PAD based on the embeddings.

Model Aggregation 134: This module aggregates the local models to update the global model in the Model Repository 131, ensuring that improvements from local training are reflected in the overall system.

Model Repository 131: This repository stores the history of models, their respective anticipated classification performance metrics, and their optimal ROC-based performance thresholds for maximum F1 scores. It also provides the VIT Embedding Head 132 and ViT Classification Tail 133 for the PAD Identification System 102.

The user interacts with multiple components in the system, providing input images, triggering the privacy-protecting inference process, submitting data to the Data Collection Module 111, and providing ground truth labels for the collected data.

A Vision Transformer (ViT) is a type of neural network that can be used for image classification and other computer vision tasks, inspired by successful transformer architecture in natural language processing. A fast, accurate, and secure (HIPAA-compliant/privacy-preserving) product has been developed that identifies any “greater than 50% narrowing” (significant stenosis) in a lower extremity artery; the AI model uses ViT technology to perform this task. The VIT architecture can include a “patch-based” regional image interpretation model paradigm. The method may combine convolutional neural network (CNN) nodal architectures with ViT technology.

The methods and systems can include a classification of maximum intensity projections (MIPs) and 3D computed tomography angiography (CTA) volumes to identify peripheral vascular disease (PVD) in patients, utilizing a privacy-protecting inference paradigm. In one embodiment, the ViT may process MIP segments of 3D CTA volumes, transforming them into numerical representations, and may classify each segment or volume for PVD using these representations. The determination of PVD at the patient level can be achieved through the classification of segments across all MIPs identified as indicative of PVD. In a second embodiment, the ViT can predict PVD by analyzing the entire 3D CTA volume rather than relying solely on MIPs generated from it. This approach can further generate regional heatmaps that provide local attribution of PVD risk, which can be superimposed and visualized in the context of the original 3D CTA or each MIP image. Additionally, the disclosure may include a privacy-protecting system that leverages model-specific numerical representations of input images, ensuring that patient data remains secure throughout the analysis process.

### Computer Systems

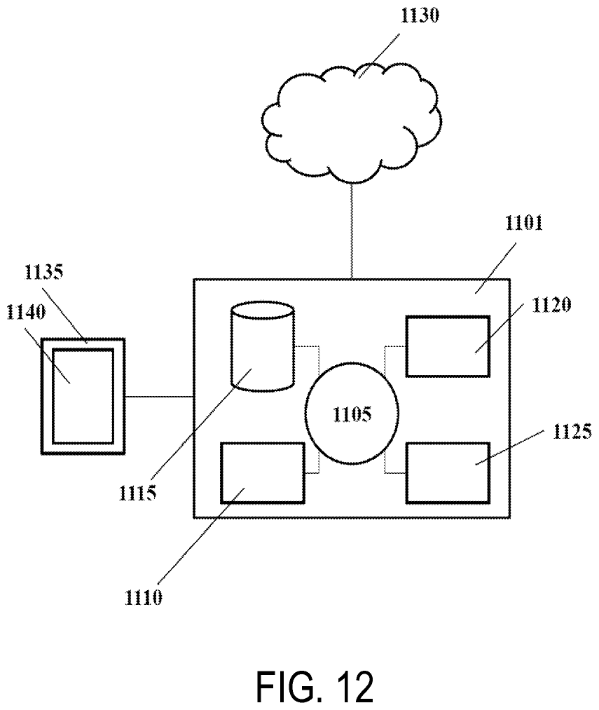

The present disclosure provides computer systems that are programmed to implement methods of the disclosure. FIG. 12 shows a computer system 1101 that is programmed or otherwise configured to process medical imaging data, apply machine learning algorithms for analysis, generate quantifiable metrics related to vascular conditions, and produce comprehensive reports to assist healthcare professionals in diagnosing and planning treatment for patients. The computer system 1101 can regulate various aspects of the methods and processes of the present disclosure, such as, for example, data acquisition, image processing, machine learning model training, and report generation. The computer system 1101 can be an electronic device of a user or a computer system that is remotely located with respect to the electronic device. The electronic device can be a mobile electronic device, such as a smartphone or tablet, capable of accessing and processing medical imaging data.

The computer system 1101 includes a central processing unit (CPU, also “processor” and “computer processor” herein) 1105, which can be a single core or multi core processor, or a plurality of processors for parallel processing. The computer system 1101 also includes memory or memory location 1110 (e.g., random-access memory, read-only memory, flash memory), electronic storage unit 1115 (e.g., hard disk), communication interface 1120 (e.g., network adapter) for communicating with one or more other systems, and peripheral devices 1125, such as cache, other memory, data storage and/or electronic display adapters. The memory 1110, storage unit 1115, interface 1120 and peripheral devices 1125 are in communication with the CPU 1105 through a communication bus (solid lines), such as a motherboard. The storage unit 1115 can be a data storage unit (or data repository) for storing data. The storage unit 1115 can also be configured as a non-transitory computer readable storage medium storing code or instructions that can be executed by the CPU 1105. The computer system 1101 can be operatively coupled to a computer network (“network”) 1130 with the aid of the communication interface 1120. The network 1130 can be the Internet, an internet and/or extranet, or an intranet and/or extranet that is in communication with the Internet. The network 1130 in some cases is a telecommunication and/or data network. The network 1130 can include one or more computer servers, which can enable distributed computing, such as cloud computing. The network 1130, in some cases with the aid of the computer system 1101, can implement a peer-to-peer network, which may enable devices coupled to the computer system 1101 to behave as a client or a server.

The CPU 1105 can execute a sequence of machine-readable instructions, which can be embodied in a program or software. The instructions may be stored in a memory location, such as the memory 1110. The instructions can be directed to the CPU 1105, which can subsequently program or otherwise configure the CPU 1105 to implement methods of the present disclosure. Examples of operations performed by the CPU 1105 can include fetch, decode, execute, and writeback.

The CPU 1105 can be part of a circuit, such as an integrated circuit. One or more other components of the system 1101 can be included in the circuit. In some cases, the circuit is an application specific integrated circuit (ASIC).

The processes and logic flows described in this specification can be performed by one or more programmable computers executing one or more computer programs to perform functions by operating on input data and generating output. The processes and logic flows can also be performed by, and apparatus can also be implemented as, special purpose logic circuitry, e.g., an FPGA (field programmable gate array), an ASIC, or a GPGPU (General purpose graphics processing unit), TPU, or other tensor or multiplication-and-accumulate processors for executing ML operations.

The storage unit 1115 can store files, such as drivers, libraries, and saved programs. The storage unit 1115 can store user data, e.g., user preferences and user programs. The computer system 1101 in some cases can include one or more additional data storage units that are external to the computer system 1101, such as located on a remote server that is in communication with the computer system 1101 through an intranet or the Internet.

The computer system 1101 can communicate with one or more remote computer systems through the network 1130. For instance, the computer system 1101 can communicate with a remote computer system of a user (e.g., a healthcare provider's workstation, a cloud-based server, or a telemedicine platform) to facilitate data sharing and collaborative analysis. Examples of remote computer systems include personal computers (e.g., portable PC), slate or tablet PC's (e.g., Apple® iPad, Samsung® Galaxy Tab), telephones, Smart phones (e.g., Apple® iPhone, Android-enabled device, Blackberry®), or personal digital assistants. The user can access the computer system 1101 via the network 1130.

Methods as described herein can be implemented by way of machine (e.g., computer processor) executable code stored on an electronic storage location of the computer system 1101, such as, for example, on the memory 1110 or electronic storage unit 1115. The machine executable or machine readable code can be provided in the form of software. During use, the code can be executed by the processor 1105. In some cases, the code can be retrieved from the storage unit 1115 and stored on the memory 1110 for ready access by the processor 1105. In some situations, the electronic storage unit 1115 can be precluded, and machine-executable instructions are stored on memory 1110.

The code can be pre-compiled and configured for use with a machine having a processer adapted to execute the code or can be compiled during runtime. The code can be supplied in a programming language that can be selected to enable the code to execute in a pre-compiled or as-compiled fashion.

Aspects of the systems and methods provided herein, such as the computer system 1101, can be embodied in programming. Various aspects of the technology may be thought of as “products” or “articles of manufacture” typically in the form of machine (or processor) executable code and/or associated data that is carried on or embodied in a type of machine readable medium. Machine-executable code can be stored on an electronic storage unit, such as memory (e.g., read-only memory, random-access memory, flash memory) or a hard disk. “Storage” type media can include any or all of the tangible memory of the computers, processors or the like, or associated modules thereof, such as various semiconductor memories, tape drives, disk drives and the like, which may provide non-transitory storage at any time for the software programming. All or portions of the software may at times be communicated through the Internet or various other telecommunication networks. Such communications, for example, may enable loading of the software from one computer or processor into another, for example, from a management server or host computer into the computer platform of an application server. Thus, another type of media that may bear the software elements includes optical, electrical, and electromagnetic waves, such as used across physical interfaces between local devices, through wired and optical landline networks and over various air-links. The physical elements that carry such waves, such as wired or wireless links, optical links or the like, also may be considered as media bearing the software. As used herein, unless restricted to non-transitory, tangible “storage” media, terms such as computer or machine “readable medium” refer to any medium that participates in providing instructions to a processor for execution.

Hence, a machine readable medium, such as computer-executable code, may take many forms, including but not limited to, a tangible storage medium, a carrier wave medium or physical transmission medium. Non-volatile storage media include, for example, optical or magnetic disks, such as any of the storage devices in any computer(s) or the like, such as may be used to implement the databases, etc. shown in the drawings. Volatile storage media include dynamic memory, such as main memory of such a computer platform. Tangible transmission media include coaxial cables; copper wire and fiber optics, including the wires that include a bus within a computer system. Carrier-wave transmission media may take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD or DVD-ROM, any other optical medium, punch cards paper tape, any other physical storage medium with patterns of holes, a RAM, a ROM, a PROM and EPROM, a FLASH-EPROM, any other memory chip or cartridge, a carrier wave transporting data or instructions, cables or links transporting such a carrier wave, or any other medium from which a computer may read programming code and/or data. Many of these forms of computer readable media may be involved in carrying one or more sequences of one or more instructions to a processor for execution.

The computer system 1101 can include or be in communication with an electronic display 1135 that includes a user interface (UI) 1140 for providing, for example, visual representations of medical imaging data, interactive tools for analyzing vascular conditions, and comprehensive reports that assist healthcare professionals in making informed decisions regarding patient diagnosis and treatment planning. Examples of UI's include, without limitation, a graphical user interface (GUI) and web-based user interface.

Methods and systems of the present disclosure can be implemented by way of one or more algorithms. An algorithm can be implemented by way of software upon execution by the central processing unit 1105. The algorithm can, for example, process medical imaging data to identify and classify vascular structures, calculate quantifiable metrics related to lesions, generate visual representations of the vascular system, and produce comprehensive reports to support clinical decision-making.

While preferred embodiments of the present disclosure have been shown and described herein, it will be obvious to those skilled in the art that such embodiments are provided by way of example only. It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

### Examples

The following illustrative examples are representative of embodiments of the applications, systems, and methods described herein and are not meant to be limiting in any way.

### Vessel Wall (VW) and Lesion Segmentations

In this case, systems and methods for diagnosing a patient having peripheral artery disease (PAD) were provided to obtain computed tomography angiography (CTA) images of the vascular system, process the CTA images to identify and classify vascular abnormalities, and generate a report that details the diagnosis. The source of data was vRad, from which approximately 4,500 CTA studies from patients were obtained. The data collection was conducted across multiple healthcare facilities, enhancing the diversity of the dataset. Additionally, the CTA images were acquired using a variety of imaging machines, ensuring variability in imaging techniques and protocols, which contributes to the robustness of the analysis. Data annotation involved receiving 266 annotated studies, which were meticulously labeled to facilitate the training and validation of the machine learning model. The annotation protocol was designed to support not only the minimum viable product (MVP) development but also future enhancements and refinements of the model.

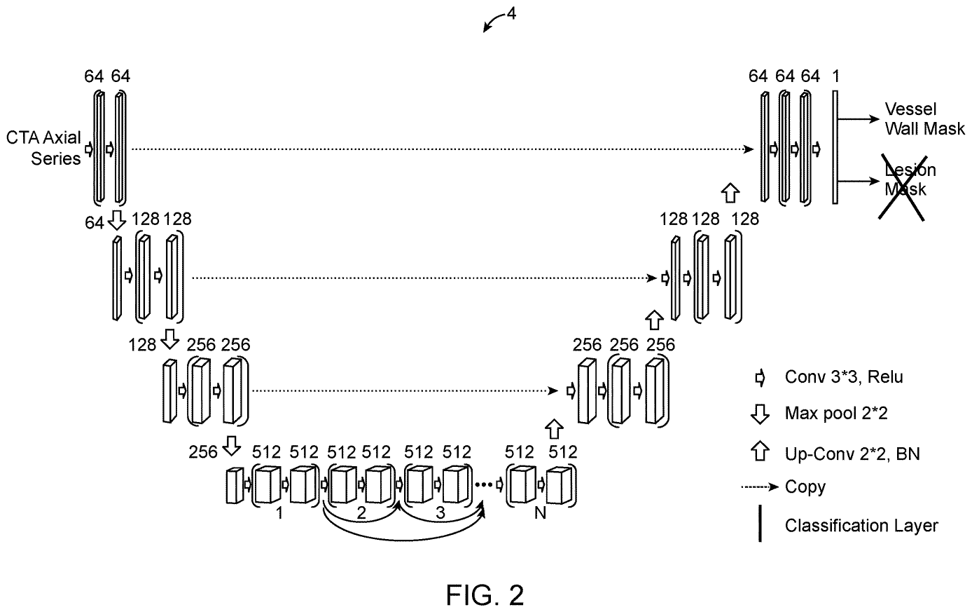

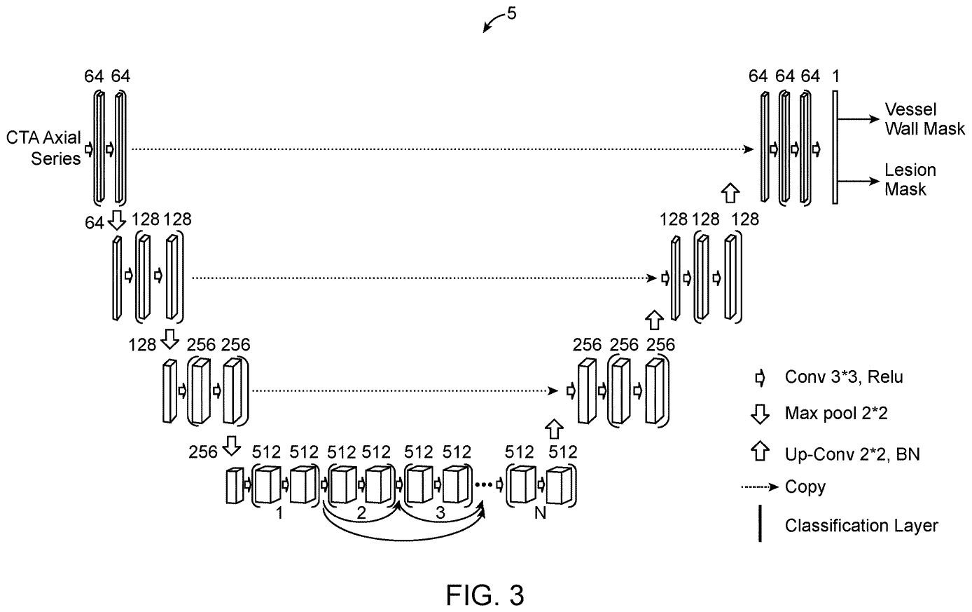

The Single-head U-Net (SHU) model 4 was utilized for vessel wall segmentation in two-dimensional (2D) imaging data, as shown in FIG. 2. This model was specifically designed to perform precise segmentation tasks by employing an encoder-decoder architecture that captured both local and global features of the vascular structures. The encoder progressively down-sampled the input images to extract high-level features, while the decoder up-sampled these features to generate accurate segmentation masks that delineated the vessel walls. The SHU model 4 was particularly effective in distinguishing between the vessel wall and surrounding tissues, enabling detailed analysis of vascular conditions. The Multi-head U-Net (MHU) model 5 was employed for vessel wall and lesion segmentation in two-dimensional (2D) imaging data, as shown in FIG. 3. This advanced model 5 featured a multi-head architecture that allowed for simultaneous processing of multiple outputs, enhancing its ability to analyze various aspects of the vascular system. Each head of the U-Net was trained to focus on different segmentation tasks, such as delineating vessel walls and identifying lesions, thereby improving the model's overall performance and accuracy.

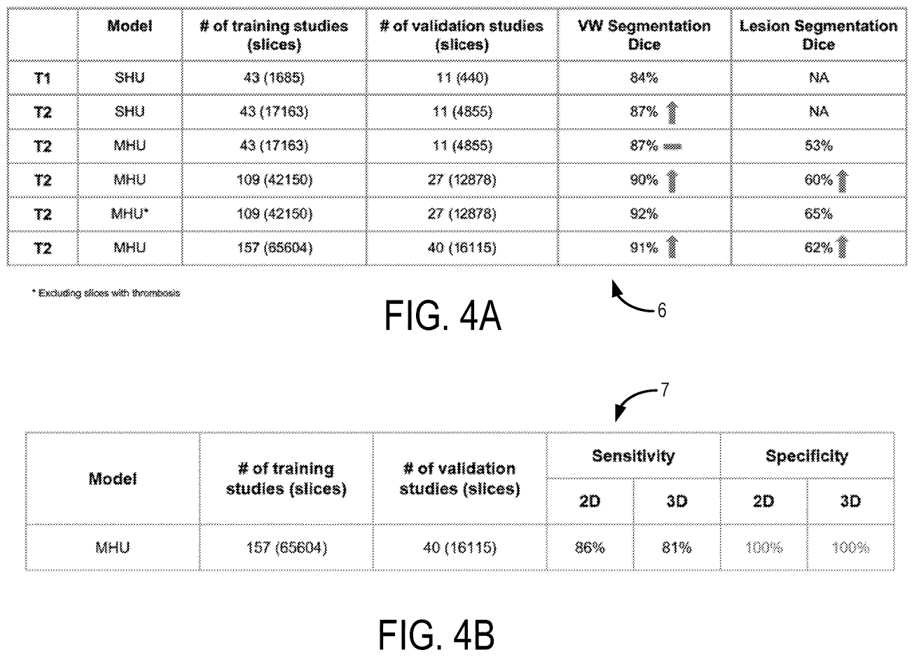

The encoder-decoder structure of the MHU captured both local and global features, with the encoder progressively down-sampling the input images to extract high-level features. The decoder then up-sampled these features to produce precise segmentation masks for both the vessel walls and lesions. By leveraging this architecture, the MHU model effectively differentiated between healthy vascular tissue and pathological features, facilitating a comprehensive assessment of vascular conditions. The processing started with the Single-head U-Net (SHU) model to segment the vessel wall only as a sanity check to ensure proper model training. This initial step is useful for verifying that the model could accurately delineate the vessel wall borders before proceeding to more complex tasks. To evaluate the model's performance, two experiments were designed. The first experiment, referred to as T1, involved training the model using slices that contained large arteries only, specifically at the level of the aorta. This focused approach ensured that the model was properly designed to learn how to segment the vessel wall border effectively. Upon achieving satisfactory results in T1, the second experiment was initiated. The second experiment, T2, involved training the model using all available slices, thereby expanding the dataset to include a wider variety of vascular structures. The Multi-head U-Net (MHU) model was trained to segment both the vessel wall and lesions using all slices. As shown in FIG. 4A, the table 6 presents the performance of different models in terms of vessel wall (VW) segmentation and lesion segmentation, evaluated using the Dice coefficient. The results are categorized by training and validation studies, with the number of slices indicated for each.

### Model T1 (SHU):

Training Studies: 43 studies (1,685 slices)
Validation Studies: 11 studies (440 slices)
VW Segmentation Dice: 84%
Lesion Segmentation Dice: Not Applicable (NA)
Analysis: The SHU model demonstrated a solid performance in vessel wall segmentation with an 84% Dice score, indicating a good level of agreement with expert annotations, although lesion segmentation was not performed in this configuration.

### Model T2 (SHU):

Training Studies: 43 studies (17,163 slices)
Validation Studies: 11 studies (4,855 slices)
VW Segmentation Dice: 87%
Lesion Segmentation Dice: NA
Analysis: The performance improved to 87% for vessel wall segmentation, suggesting that increasing the number of training slices positively impacted the model's accuracy. Lesion segmentation was still not applicable.

### Model T2 (MHU):

Training Studies: 43 studies (17,163 slices)
Validation Studies: 11 studies (4,855 slices)
VW Segmentation Dice: 87%
Lesion Segmentation Dice: 53%
Analysis: The MHU model maintained the 87% Dice score for vessel wall segmentation while introducing lesion segmentation with a Dice score of 53%. This indicates that while the model effectively segmented the vessel walls, the performance for lesion segmentation was moderate, suggesting room for improvement.

### Model T2 (MHU):

Training Studies: 109 studies (42,150 slices)
Validation Studies: 27 studies (12,878 slices)
VW Segmentation Dice: 90%
Lesion Segmentation Dice: 60%
Analysis: With an increased dataset, the MHU model achieved a 90% Dice score for vessel wall segmentation and improved lesion segmentation to 60%. This indicates that a larger training dataset significantly enhanced the model's performance in both segmentation tasks.

### Model T2 (MHU):

Training Studies: 109 studies (42,150 slices)
Validation Studies: 27 studies (12,878 slices)
VW Segmentation Dice: 92%
Lesion Segmentation Dice: 65%
Analysis: The MHU variant showed further improvement with a 92% Dice score for vessel wall segmentation and a 65% score for lesion segmentation. This suggests that adjustments by excluding slices with thrombosis contributed positively to the performance.

### Model T2 (MHU):

Training Studies: 157 studies (65,604 slices)
Validation Studies: 40 studies (16,115 slices)
VW Segmentation Dice: 91%
Lesion Segmentation Dice: 62%
Analysis: The final model configuration maintained a high Dice score of 91% for vessel wall segmentation while the lesion segmentation score was 62%. This indicates that while the model remains effective for vessel wall segmentation, the performance for lesion segmentation did not improve significantly with the additional training data. The Dice of 27% was achieved on thrombosis.

In the evaluation of the Multi-head U-Net (MHU) model, as shown in the table 7 in FIG. 4B, the training dataset included 157 studies with a total of 65,604 slices, while the validation dataset included 40 studies with 16,115 slices. The model demonstrated a sensitivity of 86% in 2D and 81% in 3D, indicating its ability to correctly identify true positive cases of vascular conditions.

There was a varying sensitivity based on the number of slices evaluated but that after training with 65k+ slices, the sensitivity was over 80% for 2D and 3D analyses. The specificity was notably high at 100% for both 2D and 3D analyses, suggesting that the model effectively identified true negative cases without misclassifying healthy structures as diseased.

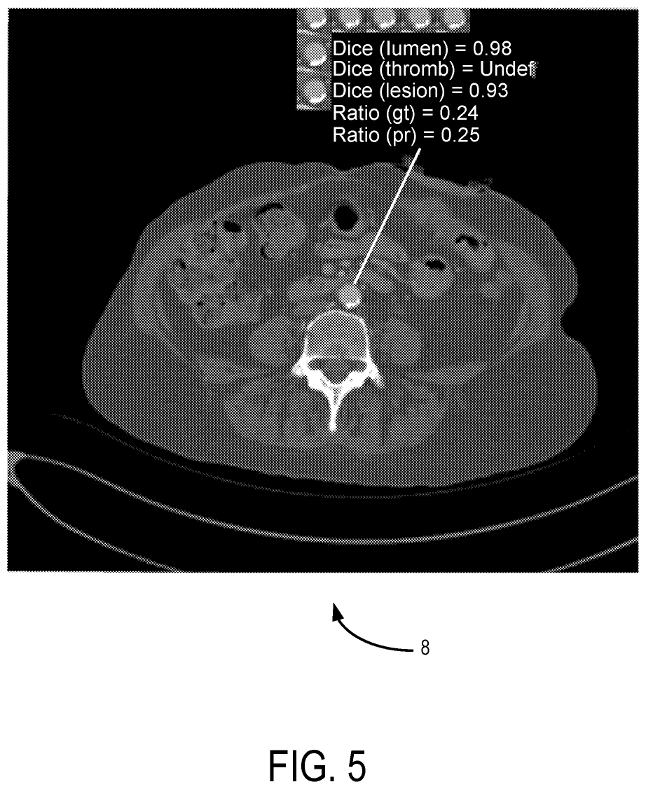

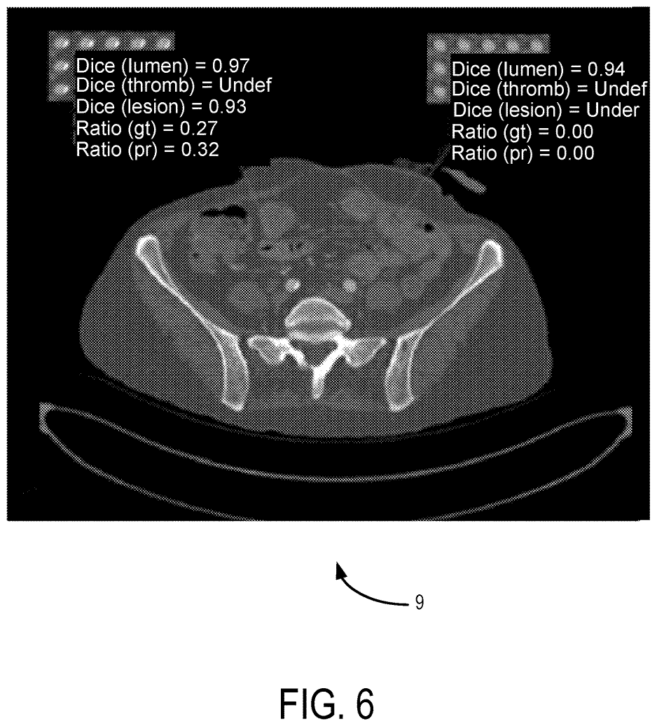

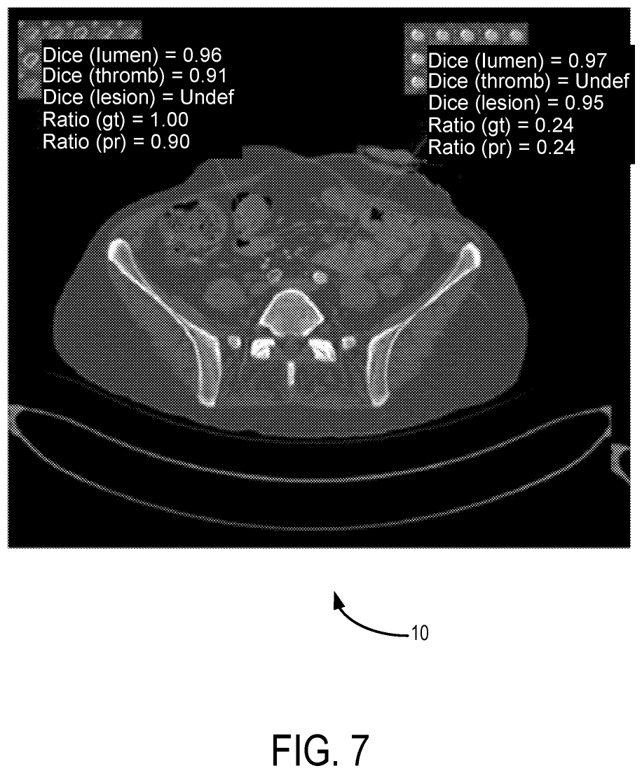

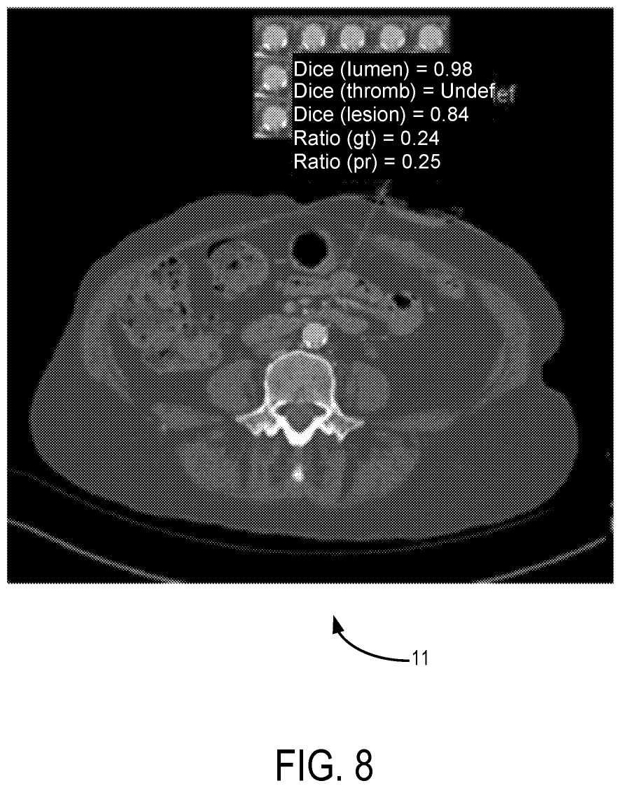

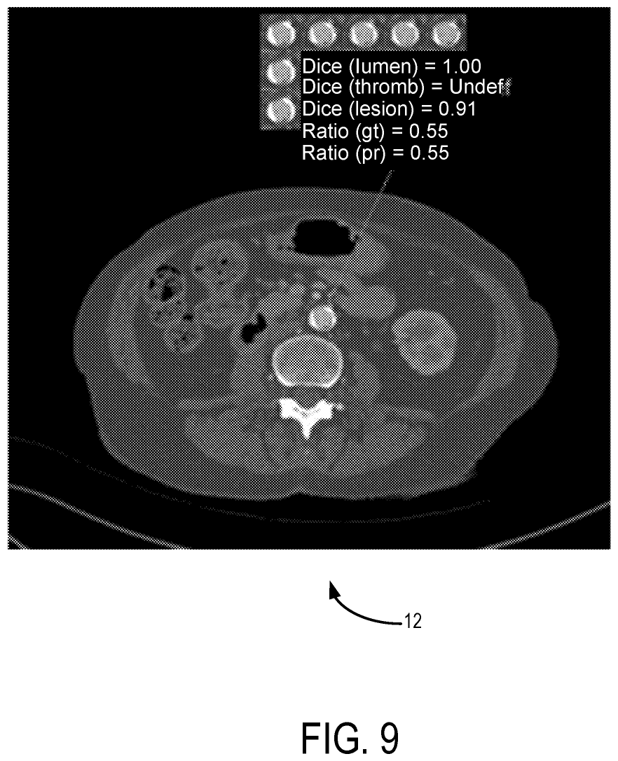

FIGS. 5-9 illustrate exemplary CTA images that represent the AI-generated vessel wall (VW) and lesion segmentation results compared to manually drawn borders by an expert. For example, FIG. 5 shows a first CTA image 8, FIG. 6 shows a second CTA image 9, FIG. 7 shows a third CTA image 10, FIG. 8 shows a fourth CTA image 11, and FIG. 9 shows a fifth CTA image 12. These figures provide a visual comparison, highlighting the accuracy and effectiveness of the machine learning model in segmenting vascular structures and lesions. The results achieved were very good despite limited training data. The model demonstrated a 92% Dice score on vessel wall segmentation, which was considered excellent, and a 62% Dice score on lesion segmentation, which was also commendable. It appeared that the model segmented lesions effectively in larger arteries (e.g., aorta). The model achieved 27% Dice for thrombosis. To enhance performance in future iterations, the model will benefit from an increase in the number of training data, the incorporation of Long Short-Term Memory (LSTM) networks to leverage information from neighboring slices, the implementation of post-processing and regularization techniques, and the performance of quality control (QC) on annotations. The extension of the Multi-head U-Net (MHU) to a multi-head bi-directional convolutional LSTM U-Net (MHLU) model will enhance the model's ability to analyze temporal information, and a landmark identification model will be developed to improve the accuracy of anatomical structure recognition.

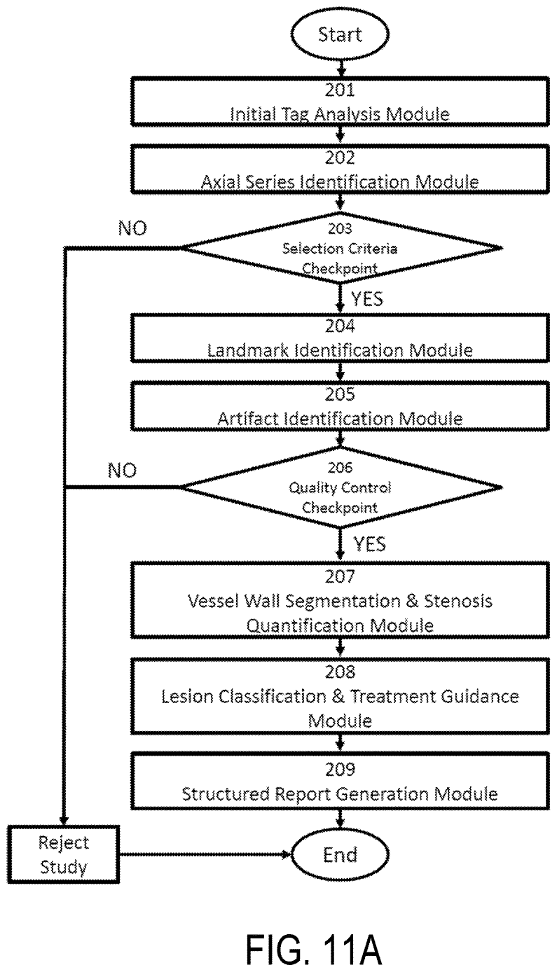

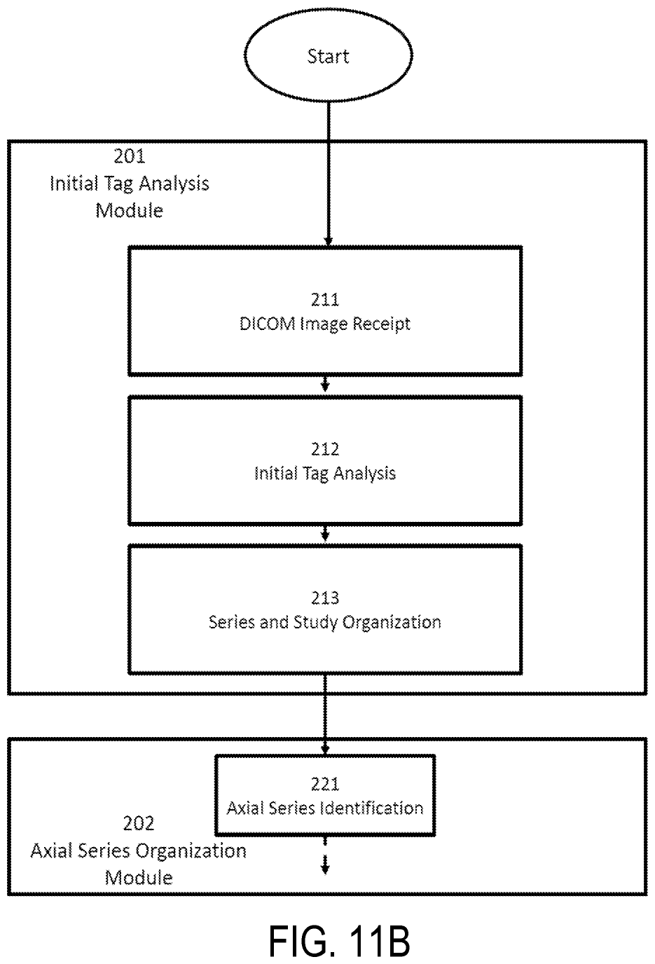

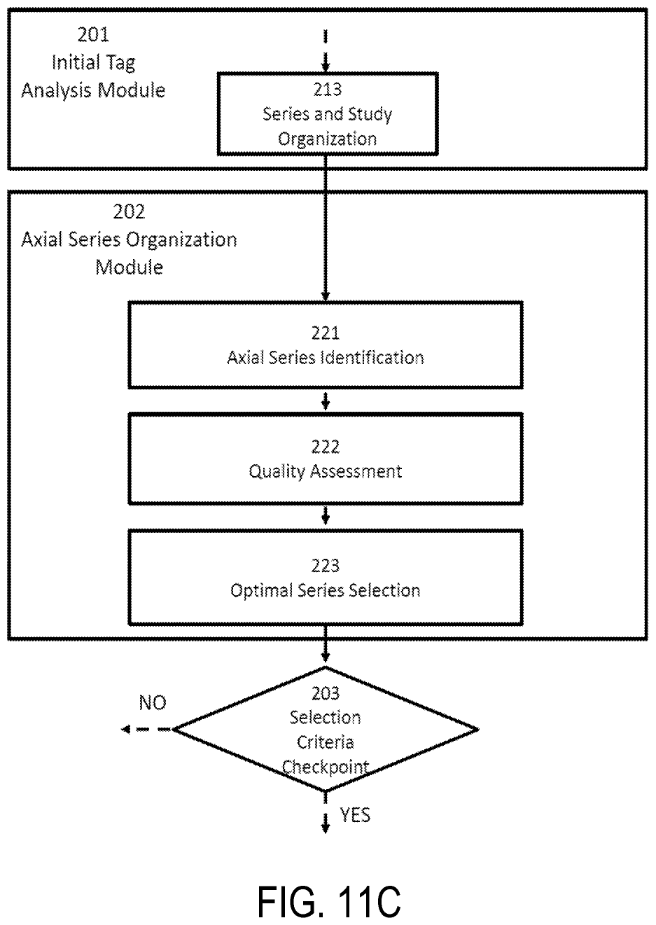

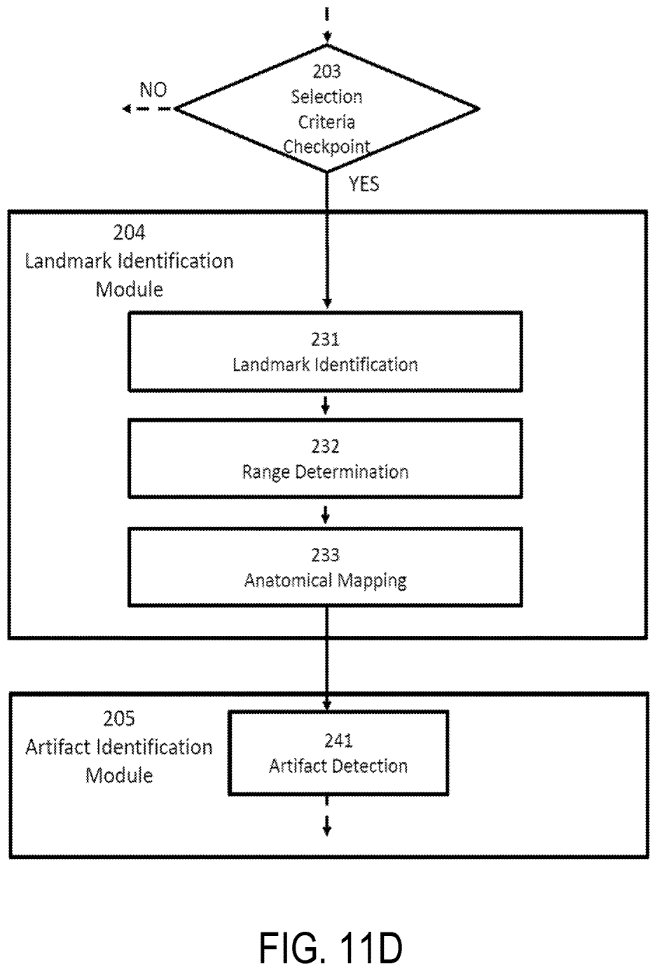

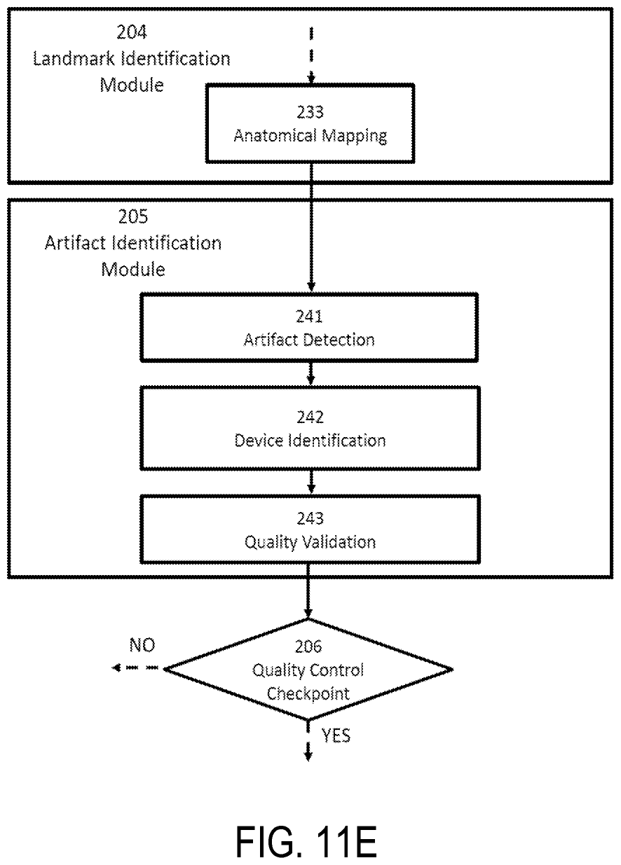

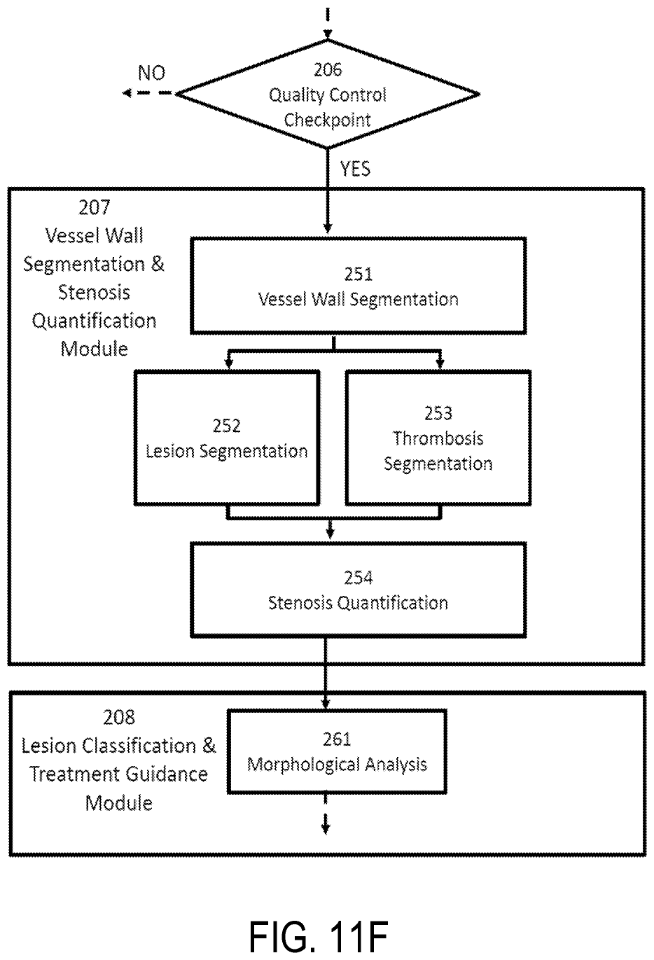

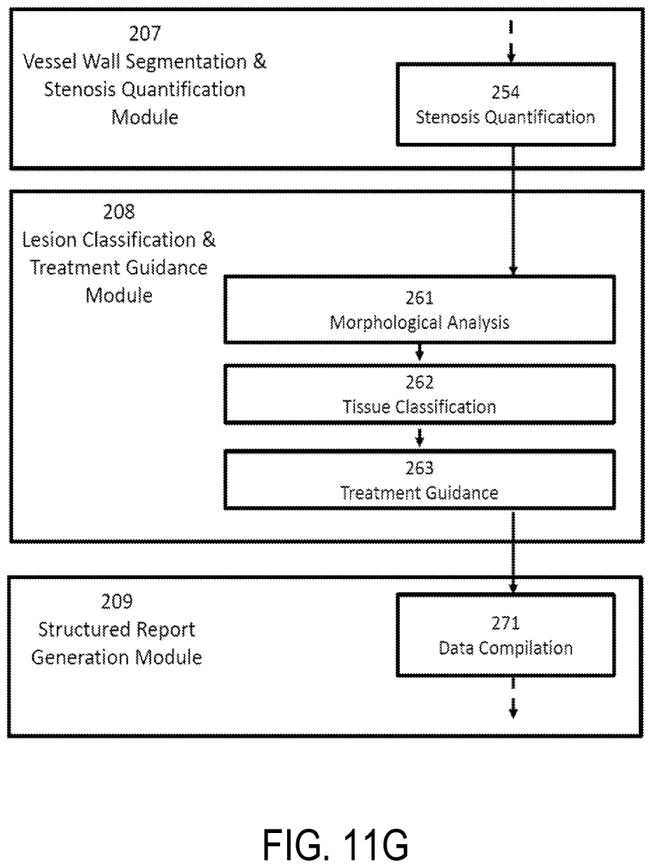

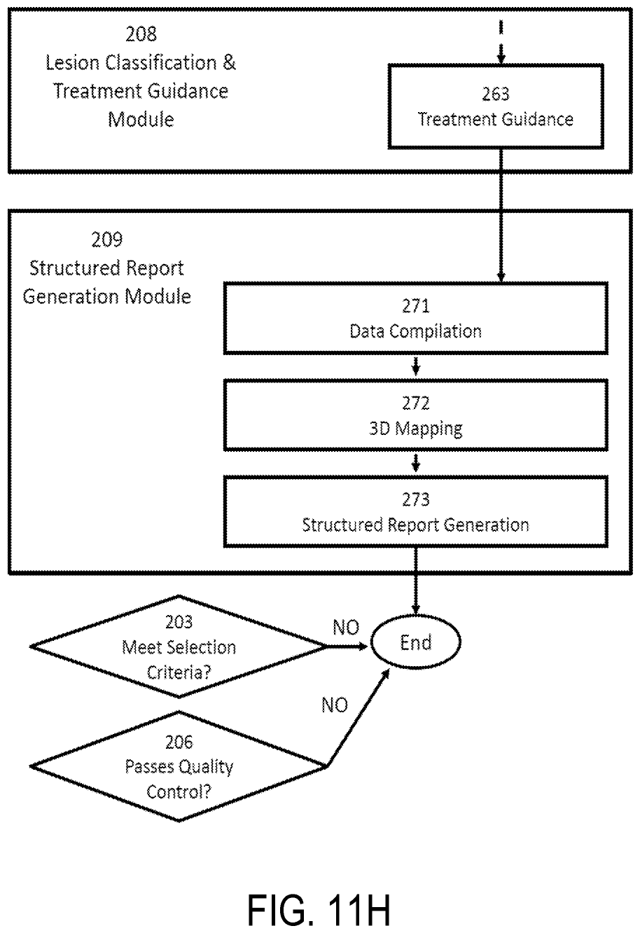

An exemplary flowchart, as depicted in FIG. 11A, illustrates exemplary sequential modules involved in the processing of medical imaging data for the classification of peripheral artery disease (PAD). As depicted in FIGS. 11B-11H, the exemplary modules include a series of exemplary steps involved in the processing of the data to classify and identify PAD.

The first phase of processing shown in FIG. 11B, Initial Tag Analysis Module 201, includes following steps: DICOM Image Receipt 211: In this step, the system receives DICOM images associated with the patient. The DICOM images are subsequently transferred to Initial Tag Analysis 212, where metadata tags are analyzed to extract relevant information about the images. The results are subsequently transferred to Series and Studies Organization 213: In this step, the images are organized into coherent series and studies for efficient processing. The organized images are subsequently transferred to Axial Series Identification 221 of the Axial Series Organization Module 202.

The second phase of processing illustrated in FIG. 11C, Axial Series Organization Module 202, includes following steps: Axial Series Identification 221: In this step, the system receives the images from Series and Studies Organization 213 and identifies axial series within the organized images. Then, the results are subsequently transferred to Quality Assessment 222, where each identified series undergoes a quality assessment to ensure it meets predefined standards. The images are subsequently transferred to Optimal Series Selection 223, where the best series are selected based on the quality assessment. At the Selection Criteria Checkpoint 203, if the selection criteria are met, the processing proceeds to the next phase. If the selection criteria are not met, the study is rejected, and the processing is terminated.

When the selection criteria are met, the images are subsequently transferred from Optimal Series Selection 223 to Landmark Identification 231, which is a part of next phase, Landmark Identification Module 204, shown in FIG. 11D. The Landmark Identification Module 204 includes following steps: Landmark Identification 231: In this step, anatomical landmarks, such as aortas, common iliac arteries, external right and left iliac arteries, left common femoral arteries, right common femoral arteries, left superficial femoral arteries, right superficial femoral arteries, left popliteal arteries, right popliteal arteries, left anterior tibial artery (ATA) bifurcation, or right ATA bifurcation are identified within the selected images. Then, the results are subsequently transferred to Range Determination 232, where the processing range for analysis is established. Then, the results are subsequently transferred to Anatomical Mapping 233: The anatomical structures, including, but not limited to, arteries, are mapped based on the identified landmarks. This targeted approach significantly mitigates the cumulative load on CPU and GPU cycles, minimizes the memory footprint by reducing the active dataset stored in RAM, and optimizes data throughput. Such efficiency helps in maintaining low latency in cloud-integrated or distributed environments, ensuring that system resources are prioritized for high-fidelity mapping and diagnostic accuracy within clinically significant regions rather than being dissipated on irrelevant anatomical structures.

The processing then proceeds to the next phase, Artifact Identification Module 205, as shown in FIG. 11E, where the images are transferred from Anatomical Mapping 233 to Artifact Detection 241. The Artifact Identification Module 205 includes following steps: Artifact Detection 241. In this step, the system detects any imaging artifacts that may affect analysis. Then, the results are subsequently transferred to Device Identification 242, where any medical devices present in the images are identified. Then, the results are subsequently transferred to Quality Validation 243, where a final quality validation is performed to ensure the integrity of the data.

At the Quality Control Checkpoint 206, if the quality control criteria are not met, the study is rejected, and the processing is terminated. If the quality control criteria are met, the processing continues to the next phase, Vessel Wall Segmentation & Stenosis Quantification Module 207, as shown in FIG. 11F, where the images are transferred from Quality Validation 243 to Vessel Wall Segmentation 251. The Vessel Wall Segmentation & Stenosis Quantification Module 207 includes following steps: Vessel Wall Segmentation 251. In this step, the model segments the vessel walls from the images. Then, the results are subsequently transferred to Lesion Segmentation 252 and/or Thrombosis Segmentation 253. At the Lesion Segmentation 252 steps, lesions within the vascular structures are identified and segmented. At the Thrombosis Segmentation 253 step, areas of thrombosis are detected and segmented. Then, the results from either or both Lesion Segmentation 252 and/or Thrombosis Segmentation 253 are subsequently transferred Stenosis Quantification 254, where a degree of stenosis is quantified based on the vessel wall segmentation data, the lesion segmentation data, and the thrombosis segmentation data.

Then, the results are subsequently transferred from Stenosis Quantification 254 to Morphological Analysis 261 of the Lesion Classification & Treatment Guidance Module 208, as shown in FIG. 11G. The Lesion Classification & Treatment Guidance Module 208 includes following steps: Morphological Analysis 261. In this step, the model analyzes the morphology of the identified lesions. Then, the results are subsequently transferred to Tissue Classification 262, where different types of tissue are classified based on their characteristics and morphology. Then, the results are subsequently transferred to Treatment Guidance 263, where recommendations for treatment are generated based on the classification and analysis.

Then, the results are subsequently transferred from Treatment Guidance 263 to Data Compilation 271 of the final phase, Structured Report Generation Module 209, as shown in FIG. 11H. The Structed Report Generation Module 209 includes following steps: Data Compilation 271. In this step, all relevant data and findings are compiled into a cohesive format. Then, the results are subsequently transferred to 3D Mapping 272, where a 3D map of the vascular structures is generated. Then, the results are subsequently transferred to Structured Report Creation 273, where a structured report is created, summarizing the findings and recommendations for clinical use.

### Graph-Based Segmentation Pruning

In some embodiments, a deep learning-based semantic segmentation algorithm can be utilized to generate a series of 2D segmentation masks for lumen, lesions, and thrombosis. The input image may contain, for example, 1 or 2 arteries of interest. As a result, the output lumen mask may include of, for example, 1 or 2 connected components. In some embodiments, false positive regions that may not be part of the lumen can be generated by the algorithm. An algorithm can be proposed that processes the generated 2D lumen segmentation masks to remove these false positive regions. This algorithm can employ a graph-based approach to identify the smoothest 3D segmentation mask, which may correspond to the most probable vessel path. In some embodiments, a connected component (CC) labeling algorithm can be applied to each 2D slice to generate CCs. A graph can be constructed based on the CCs across all slices, with each CC considered a node of the graph. Edges can be created between CCs of one slice and the CCs of the nearest slice (most likely the next slice) that contains CCs. The weight of each edge can be defined as a weighted combination of the Euclidean distance between the centers of mass and the Dice score of the two regions, with smaller weights assigned to more similar regions. To achieve a smooth segmentation outcome (most probable vessel path), a graph traversal algorithm can be utilized to obtain a list of edges with minimum weight. For example, the minimum spanning tree (MST) can be generated, which represents a subset of the edges of a connected, edge-weighted undirected graph that connects all the vertices together without any cycles and with the minimum possible total edge weight. As the generated tree may still contain false positive regions, a post-processing algorithm can be employed to remove the false positive (orphaned) nodes. For example, nodes that are connected to only one other node (except for the nodes of the first and last slices) can be repeatedly removed until no nodes remain to be removed. In some embodiments, Dijkstra's Algorithm can be used. The Dijkstra's Algorithm can find the shortest weighted path. The Dijkstra's Algorithm may be applied separately for the slices before and after bifurcation. The graph-based segmentation pruning can include following steps:

### Step 1: Construct a Graph Representation

In some embodiments, each segmented vessel region in a 2D slice can be treated as a node, and edges can be formed based on spatial proximity across slices.

Sᵢ={Rᵢ₁, Rᵢ₂, . . . } may represent the set of segmented regions in slice i, where each Rᵢⱼ may denote an individual connected component (a vessel segment). A graph G(V, E) can be defined where:

Nodes V correspond to segmented regions across all slices.

Edges E connect vessel regions in adjacent slices (or the nearest slice containing regions) based on proximity and shape similarity.

The graph structure may allow for the representation of relationships between vessel segments.

### Step 2: Define Edge Weights

In some embodiments, to ensure connectivity between the correct vessel segments, a weight W can be assigned to each edge based on a combination of the following factors:

Euclidean Distance (dᵢⱼ): The center-of-mass distance between two regions in adjacent slices.

Dice Score (Dᵢⱼ): A measure of similarity between the two regions.

The edge weight can be computed using the formula: wᵢⱼ=αdᵢⱼ−βDᵢⱼ
where α and β are tuning parameters that can be adjusted to balance the influence of distance and similarity on the edge weight. The edge weighted approach may allow for a more nuanced representation of the relationships between vessel segments.

### Step 3: Find the Most Probable Vessel Path

In some embodiments, graph traversal algorithms can be utilized to connect disjoint vessel components. These algorithms may enhance the connectivity and navigability of the graph representing the vascular system.

Minimum Spanning Tree (MST): The algorithm may help ensure that all vessel components are connected with minimal added connections. By selecting edges with the lowest weights, the MST may guarantee that the entire graph remains connected while minimizing the total edge weight.

Dijkstra's Algorithm: The algorithm can be employed to find the shortest weighted path, for example from the aorta down to the legs. By calculating the shortest paths based on the assigned edge weights, Dijkstra's Algorithm can facilitate the identification of the most efficient route through the vascular network, ensuring that the traversal reflects the best or one of the best path for blood flow or other analyses.

### Step 4: Remove False Positives

In some embodiments, after constructing the vessel paths, the removal of orphaned nodes that are not part of the main vessel tree may be performed. Orphaned nodes refer to those that lack connections to the primary vascular structure, often arising from segmentation errors, noise, or false positives. Eliminating these nodes can improve the accuracy of the vascular representation, ensuring that the model accurately reflects the true anatomy of the vessels. Additionally, a cleaner graph may enhance clarity for visualization. The process may involve identifying orphaned nodes, typically those connected to only one other node or completely isolated, followed by their removal to streamline the graph. Finally, validating the remaining structure may ensure that desired vessel components remain connected.

It shall be understood that different aspects of the disclosure can be appreciated individually, collectively, or in combination with each other. Various aspects of the disclosure described herein may be applied to any of the particular applications disclosed herein. The compositions of matter disclosed herein in the composition section of the present disclosure may be utilized in the method section including methods of use and production disclosed herein, or vice versa.

As used herein, the term “at least one of” can refer to and encompass any and all possible combinations of one or more of the associated listed terms. For example, the term “at least one of A, B, or C” means that (i) at least one of A, (ii) at least one of B, (iii) at least one of C, (iv) at least one of A and at least one of B, (v) at least one of B and at least one of C, (vi) at least one of A and at least one of C, or (vi) at least one of A, at least one of B and at least one of C are possible, where A, B and C may be singular or plural.

While this specification contains many specific implementation details, these should not be construed as limitations on the scope of any invention or on the scope of what may be claimed, but rather as descriptions of features that may be specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations, one or more features from a combination can in some cases be excised from the combination, and the combination may be directed to a subcombination or variation of a subcombination.

It is not intended that the disclosure be limited by the specific examples provided within the specification. While the disclosure has been described with reference to the aforementioned specification, the descriptions and illustrations of the embodiments herein are not meant to be construed in a limiting sense. Numerous variations, changes, and substitutions will now occur to those skilled in the art without departing from the disclosure. Furthermore, it shall be understood that all aspects of the disclosure are not limited to the specific depictions, configurations or relative proportions set forth herein which depend upon a variety of conditions and variables. It should be understood that various alternatives to the embodiments of the disclosure described herein may be employed in practicing the disclosure. It is therefore contemplated that the disclosure shall also cover any such alternatives, modifications, variations, or equivalents. It is intended that the following claims define the scope of the disclosure and that methods and structures within the scope of these claims and their equivalents be covered thereby.

1. A method for identifying treatments for a patient having peripheral artery disease (PAD), the method comprising:
obtaining at least one computed tomography angiography (CTA) image of at least one portion of the patient, wherein the at least one CTA image of the at least one portion of the patient comprises at least one portion of a vascular system;
providing the at least one CTA image of the at least one portion of the patient as input to a machine learning model, wherein the machine learning model is a trained machine learning model trained by a database of at least one reference image with known characteristics associated with PAD;
classifying characteristics of the at least one portion of the vascular system based on the at least one CTA image of the at least one portion of the patient using the trained machine learning model, wherein the trained machine learning model is configured to characterize the at least one portion of the vascular system;
determining a diagnosis of the patient based on the classified characteristics of the at least one portion of the vascular system from the at least one CTA image of the at least one portion of the patient, wherein the diagnosis comprises at least one condition of the patient correlated to at least one indicator of PAD; and
generating a report based on the diagnosis, wherein the report comprises a diagnosis of PAD, a severity of PAD, and/or recommendations for treatment options.

2. The method of claim 1, wherein classifying the characteristics comprises generating one or more groups of images from the at least one CTA image based on characteristics associated with PAD, and wherein the one or more groups of images comprises segmented images highlighting vascular abnormalities, annotated images, and/or comparative images.

3. The method of claim 2, wherein classifying the characteristics comprises matching images from the one or more groups of images to the at least one reference image with known characteristics associated with PAD, wherein a process of matching images is conducted by the trained machine learning model.

4. The method of claim 1, wherein obtaining the at least one CTA image comprises receiving at least one Digital Imaging and Communications in Medicine (DICOM) image, wherein the at least one DICOM image is associated with the patient having PAD.

5. The method of claim 1, wherein the machine learning model comprises a Vision Transformer (ViT) model.

6. The method of claim 5, wherein the ViT model is configured to process maximum intensity projections (MIP) segments of 3D CTA volumes, wherein the MIP segments comprise numerical transformed representations of at least one of local features or global features derived from the 3D CTA volumes, and wherein the VIT model is configured to analyze the 3D CTA volumes and generate regional heatmaps, wherein the regional heatmaps are attributed to vascular abnormalities.

7. The method of claim 1, wherein the trained machine learning model is configured to automatically identify and label anatomical structures, wherein the anatomical structures comprise aorta, common iliac arteries, external right and left iliac arteries, left common femoral arteries, right common femoral arteries, left superficial femoral arteries, right superficial femoral arteries, left popliteal arteries, right popliteal arteries, left anterior tibial artery (ATA) bifurcation, or right ATA bifurcation.

8. The method of claim 1, wherein the trained machine learning model is configured to identify medical devices within the at least one portion of the vascular system associated with the at least one CTA image of the at least one portion of the patient, wherein the medical devices comprise stents or bypass grafts.

9. The method of claim 1, wherein the trained machine learning model is configured to digitally segment regions within at least one image of the at least one portion of the vascular system associated with the at least one CTA image of the at least one portion of the patient, wherein segmented image comprise arterial walls, lesions, or thrombosis.

10. The method of claim 9, wherein the trained machine learning model is configured to provide a treatment guidance based on a classification of a lesion's morphology, wherein the treatment guidance is selected from a set of recorded treatments according to the classification of the lesion's morphology, wherein the recorded treatments are approved by certified clinicians.

11. The method of claim 10, wherein the trained machine learning model is configured to provide a guidance on selecting an intervention device based on the classification of at least one of the lesion's morphology or lesions characteristics, wherein the guidance on selecting the intervention device is selected from a set of recorded guidance, wherein the recorded guidance is approved by certified clinicians.

12. The method of claim 11, wherein the trained machine learning model is configured to generate a structured radiological report comprising at least one of one or more conditions correlated to at least one indicator of PAD, a 3D vascular mapping, detailed lesion characterization, quantitative measurements or treatment recommendations.

13. The method of claim 1, wherein the trained machine learning model comprises one or more single-head U-Net (SHU) and/or multi-head U-Net (MHU) convolutional layers.

14. The method of claim 1, wherein the trained machine learning model is configured to recommend a treatment plan, wherein the treatment plan comprises a recommended use of an interventional tool based on a 3D construction of lesions identified within the vascular system.

15. A system, comprising:
at least one processor; and
at least one memory device coupled with the at least one processor, the at least one memory device comprising instructions, wherein execution of the instructions causes the at least one processor to perform operations comprising:
obtaining at least one computed tomography angiography (CTA) image of at least one portion of a patient, wherein the at least one CTA image of the at least one portion of the patient comprises at least one portion of a vascular system;
providing the at least one CTA image of the at least one portion of the patient as input to a machine learning model, wherein the machine learning model is a trained machine learning model trained by a database of at least one reference image with known characteristics associated with peripheral artery disease (PAD);
classifying characteristics of the at least one portion of the vascular system based on the at least one CTA image of the at least one portion of the patient using the trained machine learning model, wherein the trained machine learning model is configured to characterize the at least one portion of the vascular system;
determining a diagnosis of the patient based on the classified characteristics of the at least one portion of the vascular system from the at least one CTA image of the at least one portion of the patient, wherein the diagnosis comprises at least one condition of the patient correlated to at least one indicator of PAD; and
generating a report based on the diagnosis, wherein the report comprises a diagnosis of PAD, a severity of PAD, and/or recommendations for treatment options.

16. The system of claim 15, wherein classifying the characteristics comprises generating one or more groups of images from the at least one CTA image based on characteristics associated with PAD, and wherein generating the one or more groups of images comprises segmented images highlighting vascular abnormalities, annotated images, and/or comparative images.

17. The system of claim 16, wherein classifying the characteristics comprises matching images from the one or more groups of images to the at least one reference image with known characteristics associated with PAD, wherein a process of matching images is conducted by the trained machine learning model.

18. The system of claim 15, wherein obtaining the at least one CTA image comprises receiving at least one Digital Imaging and Communications in Medicine (DICOM) image, wherein the at least one DICOM image is associated with the patient having PAD.

19. The system of claim 15, wherein the machine learning model comprises a Vision Transformer (ViT) model.

20. The system of claim 19, wherein the ViT model is configured to process maximum intensity projections (MIP) segments of 3D CTA volumes, wherein the MIP segments comprise numerical transformed representations of at least one of local features or global features derived from the 3D CTA volumes, and wherein the VIT model is configured to analyze the 3D CTA volumes and generate regional heatmaps, wherein the regional heatmaps are attributed to vascular abnormalities.

21. The system of claim 15, wherein the trained machine learning model is configured to automatically identify and label anatomical structures, wherein the anatomical structures comprise aorta, common iliac arteries, external right and left iliac arteries, left common femoral arteries, right common femoral arteries, left superficial femoral arteries, right superficial femoral arteries, left popliteal arteries, right popliteal arteries, left anterior tibial artery (ATA) bifurcation, or right ATA bifurcation.

22. The system of claim 15, wherein the trained machine learning model is configured to identify medical devices within the at least one portion of the vascular system associated with the at least one CTA image of the at least one portion of the patient, wherein the medical devices comprise stents or bypass grafts.

23. The system of claim 15, wherein the trained machine learning model is configured to digitally segment regions within at least one image of the at least one portion of the vascular system associated with the at least one CTA image of the at least one portion of the patient, wherein segmented image comprise arterial walls, lesions, or thrombosis.

24. The system of claim 15, wherein the trained machine learning model is configured to provide a treatment guidance based on a classification of a lesion's morphology, wherein the treatment guidance is selected from a set of recorded treatments according to the classification of the lesion's morphology, wherein the recorded treatments are approved by certified clinicians.

25. The system of claim 15, wherein the trained machine learning model is configured to provide a guidance on selecting an intervention device based on a classification of at least one of a lesion's morphology or a lesion's characteristics, wherein the guidance on selecting the intervention device is selected from a set of recorded guidance, wherein the recorded guidance is approved by certified clinicians.

26. The system of claim 15, wherein the trained machine learning model is configured to generate a structured radiological report comprising at least one of one or more conditions correlated to at least one indicator of PAD, a 3D vascular mapping, detailed lesion characterization, quantitative measurements or treatment recommendations.

27. The system of claim 15, wherein the trained machine learning model comprises one or more single-head U-Net (SHU) and/or multi-head U-Net (MHU) convolutional layers.

28. The system of claim 15, wherein the trained machine learning model is configured to recommend a treatment plan, wherein the treatment plan comprises a recommended use of an interventional tool based on a 3D construction of lesions identified within the vascular system.

29. A non-transitory computer readable storage medium storing instructions that when executed by one or mor processors cause the one or more processors to perform operations for identifying treatments for a patient having peripheral artery disease (PAD), the operations comprising:
obtaining at least one computed tomography angiography (CTA) image of at least one portion of the patient, wherein the at least one CTA image of the at least one portion of the patient comprise at least one portion of a vascular system;
providing the at least one CTA image of the at least one portion of the patient as input to a machine learning model, wherein the machine learning model is a trained machine learning model trained by a database of at least one reference image with known characteristics associated with PAD;
classifying characteristics of the at least one portion of the vascular system based on the at least one CTA image of the at least one portion of the patient using the trained machine learning model, wherein the trained machine learning model is configured to characterize the at least one portion of the vascular system;
determining a diagnosis of the patient based on the classified characteristics of the at least one portion of the vascular system from the at least one CTA image of the at least one portion of the patient, wherein the diagnosis comprises at least one condition of the patient correlated to at least one indicator of PAD; and
generating a report based on the diagnosis, wherein the report comprises a diagnosis of PAD, a severity of PAD, and/or recommendations for treatment options.

30. The non-transitory computer readable storage medium of claim 29, wherein the trained machine learning model is configured to provide a treatment guidance based on the classification of a lesion's morphology, wherein the treatment guidance is selected from a set of recorded treatments according to the classification of the lesion's morphology, wherein the recorded treatments are approved by certified clinicians.
